(19) United States
(12) Patent Application Publication
(10) Pub. No.: US 2024/0423904 A1
(43) Pub. Date: Dec. 26, 2024

(54) Systems and Methods for Controlling Chemical Reactions Using Ultrasound

(71) Applicant: California Institute of Technology, Pasadena, CA (US)

(72) Inventors: Maxwell J. Robb, Altadena, CA (US); Mikhail Georgievich Shapiro, Los Angeles, CA (US); Yuxing Yao, Pasadena, CA (US); Molly E. McFadden, Walnut Creek, CA (US)

(73) Assignee: California Institute of Technology, Pasadena, CA (US)

(21) Appl. No.: 18/734,977

(22) Filed: Jun. 5, 2024

Related U.S. Application Data
(60) Provisional application No. 63/472,767, filed on Jun. 13, 2023.

(51) Int. Cl. A61K 9/00; A61K 31/4745; A61K 41/00; A61K 47/32

(52) U.S. Cl. CPC A61K 9/0009; A61K 31/4745; A61K 41/0033; A61K 47/32

(57) ABSTRACT

Systems and methods for activating mechanochemical reactions remotely using biocompatible ultrasound in the presence of gas-filled structures are described. The collapse of gas-filled structures is achieved using biocompatible ultrasound. In turn, collapse of the gas-filled structures can mechanochemically activate mechanophore-functionalized polymers in solution. Mechanochemical activation of mechanophore-functionalized polymers under physiological conditions can trigger the release and/or delivery of a variety of cargos with the spatial and temporal precision and deep tissue penetration afforded by focused ultrasound.

### CROSS-REFERENCE TO RELATED APPLICATIONS

The current application claims the benefit of and priority under 35 U.S.C. § 119 (e) to U.S. Provisional Patent Application No. 63/472,767 entitled “Method For Controlling Chemical Reactions Using Ultrasound” filed Jun. 13, 2023. The disclosure of U.S. Provisional Patent Application No. 63/472,767 is hereby incorporated by reference in its entirety for all purposes.

### FIELD OF THE INVENTION

The present invention generally relates to systems and methods for controlling chemical reactions using ultrasound.

### BACKGROUND OF THE INVENTION

Ultrasound is sound with frequencies greater than 20 kilohertz (kHz). This frequency is the approximate upper audible limit of human hearing. Ultrasonic devices operate with frequencies from 20 kHz up to several gigahertz. Ultrasound can be used in many different fields. Ultrasonic devices are used to detect objects and measure distances. Ultrasound imaging or sonography is often used in medicine. In the nondestructive testing of products and structures, ultrasound is used to detect invisible flaws. Industrially, ultrasound is used for cleaning, mixing, and accelerating chemical processes. Animals such as bats and porpoises use ultrasound for locating prey and obstacles.

Focused ultrasound uses an acoustic lens to concentrate multiple intersecting beams of ultrasound on a target deep in the body with high precision and accuracy. Depending on the design of the lens and the ultrasound parameters, the target can have dimensions in the millimeter ranges and centimeter ranges.

### BRIEF SUMMARY OF THE INVENTION

Many embodiments are directed to systems and methods for activating mechanochemical reactions using biocompatible ultrasound. In some embodiments, the biocompatible ultrasound can control mechanochemical reactions under physiological conditions remotely with spatial and temporal precision. Several embodiments implement synergistic systems that can cause the collapse of gas-filled structures such as (but not limited to) gas vesicles and/or microbubbles using biocompatible ultrasound. In turn, collapse of the gas-filled structures can mechanically activate the chemical reaction of mechanophore-functionalized polymers in solution. The gas-filled structures function as seeds for gas-filled structures formation and cavitation upon treatment with biocompatible ultrasound, which is effectively coupled to the mechanochemical activation of mechanophore-functionalized polymers.

In many embodiments, mechanophores can be any stress-sensitive molecules that exhibit selective reactivity under mechanical forces. Several embodiments can activate various types of mechanophores using ultrasound via gas-filled structures. Ultrasound in accordance with some embodiments applies mechanical forces to activate chemical transformations of mechanophores to produce a wide range of functional responses. Examples of various types of mechanophores include (but are not limited to) mechanophores with covalently attached cargo molecules and/or payloads, mechanochromic mechanophores, derivatives of cyclobutane, benzocyclobutene, dihalocyclopropranes, and/or Diels-Alder adducts. In some embodiments, ultrasound can activate mechanochemical reactions by triggering the release of cargo molecules attached to the mechanophores. The cargo molecules can have various functions and can be released by ultrasound with spatial and temporal control. In some embodiments, ultrasound can activate mechanochromic mechanophores (also known as color-changing mechanophores). The mechanochromic mechanophores can change colors or luminescence when a mechanical force is applied to the mechanophores such that it enables applications such as (but not limited to) force sensing. Examples of mechanochromic mechanophores include (but are not limited to) spiropyran, derivatives of spiropyran, naphthopyran, benzopyran, rhodamine, oxazine, triarylmethane, and/or diarylbibenzofuranone. In some embodiments, ultrasound can activate mechanophores such as (but not limited to) ladderenes to change and/or modulate their electrical conductivity. In some embodiments, ultrasound can activate mechanophores such as (but not limited to) benzocyclobutene, beta-lactams to reveal reactive groups. As can be readily appreciated, any of a variety of mechanophores can be activated via ultrasound as appropriate to the requirements of specific applications in accordance with various embodiments.

In many embodiments, the mechanochemical activation of mechanophore-containing polymers under physiological conditions can trigger the release and/or deliver a variety of cargos. The cargo molecules can include (but are not limited to) small molecules, proteins, polypeptides, nucleic acids, pharmaceutical molecules, catalysts, fluorescent molecules, luminescent probes, molecules that can change color upon release, and any combinations thereof. The remote-controlled and targeted release of desired molecules under biocompatible conditions in accordance with many embodiments enables diverse biological and biomedical applications such as (but not limited to) diagnostics, detection, therapeutics, imaging, and/or pharmaceuticals. The applications can be in vitro and/or in vivo for human and non-human subjects.

Some embodiments include a method for activating a mechanochemical reaction comprising: applying ultrasound to a solution at least containing a concentration of at least one polymer and a plurality of gas-filled structures; wherein each of the at least one polymer comprises a polymer chain functionalized with at least one mechanophore; and wherein the ultrasound is applied such that the plurality of gas-filled structures collapses thereby transducing the ultrasound to a mechanical force, such that the mechanical force activates the mechanochemical reaction to produce a functional response from the at least one mechanophore.

In some embodiments, the at least one mechanophore is selected from the group consisting of: a mechanochromic mechanophore, a mechanophore with a cargo molecule, a mechanophore that changes electrical conductivity, and a mechanophore that reveals at least one reactive group.

In some embodiments, the function response from the at least one mechanophore is selected from the group consisting of: changing color, changing luminescence, releasing the cargo molecule, changing electrical conductivity, and revealing the at least one reactive group.

In some embodiments, the at least one mechanophore is selected from the group consisting of: spiropyran, a derivative of spiropyran, naphthopyran, benzopyran, rhodamine, oxazine, triarylmethane, diarylbibenzofuranone, a derivative of cyclobutane, benzocyclobutene, dihalocycloproprane, a Diels-Alder adduct, a beta-lactam, ladderane, and masked 2-furylcarbinol.

In some embodiments, the at least one mechanophore is attached to the polymer chain via: a covalent bond, bioconjugation, or click chemistry.

In some embodiments, the at least one mechanophore comprises at least one cargo molecule selected from the group consisting of: a macromolecule, a small molecule, a micromolecule, an organic molecule, an inorganic molecule, an amino acid, a polypeptide, a protein, a nucleic acid, a DNA, an RNA, a monosaccharide, a polysaccharide, and any combinations thereof.

In some embodiments, the at least one mechanophore comprises at least one cargo molecule selected from the group consisting of: a drug, a chemotherapeutic drug, a catalyst, a fluorescent molecule, a fluorescent probe, a fluorophore, and a luminescent molecule.

In some embodiments, the ultrasound is a focused ultrasound.

In some embodiments, the polymer chain is poly(2-(methylsulfinyl)ethyl acrylate), the mechanophore is a masked 2-furylcarbinol mechanophore, and an anticancer drug camptothecin is covalently attached to the mechanophore.

In some embodiments, the solution is selected from the group consisting of: an aqueous solution, an organic solution, a buffer solution, an intercellular environment, an intracellular environment, an in situ environment, an in vitro environment, an in vivo environment, a physiological environment, and a clinically relevant environment.

In some embodiments, each of the plurality of gas-filled structures is selected from the group consisting of: a gas vesicle, a natural gas vesicle, a synthetic gas vesicle, a microbubble, and any combinations thereof.

In some embodiments, each of the plurality of gas-filled structures comprises a gas selected from the group consisting of: a non-reactive gas, an inert gas, air, nitrogen, carbon dioxide, helium, argon, neon, xenon, and any combinations thereof.

In some embodiments, each of the plurality of gas-filled structures has an average diameter from 50 nm to 10 microns.

In some embodiments, each of the plurality of gas-filled structures has an average length from 50 nm to 10 microns.

In some embodiments, the plurality of gas-filled structures comprises a plurality of gas vesicles, and the plurality of gas vesicles has an average diameter from 45 nm to 250 nm and an average length from 100 nm to 600 nm.

In some embodiments, the ultrasound causes a temperature increase of the solution less than or equal to 5° C.

In some embodiments, the ultrasound has an acoustic intensity Isppa (spatial-peak-pulse-average) less than or equal to 1000 W/cm².

In some embodiments, the ultrasound has an acoustic intensity Iₛₚₜₐ (spatial-peak-time-average) less than or equal to 50 W/cm².

In some embodiments, the ultrasound has an acoustic intensity Iₛₚₚₐ less than or equal to 80 W/cm² and Iₛₚₜₐ less than or equal to 4 W/cm².

Some embodiments include a method for delivering one or more cargo molecules comprising: applying focused ultrasound to an aqueous environment at least containing a concentration of at least one polymer and a plurality of gas-filled structures; wherein each of the at least one polymer comprises a polymer chain functionalized with at least one mechanophore, and wherein the at least one mechanophore comprises at least one cargo molecule; and wherein the focused ultrasound is applied such that the plurality of gas-filled structures collapses thereby transducing the focused ultrasound to a mechanical force, such that the mechanical force activates the at least one mechanophore to release the at least one cargo molecule into the aqueous environment.

In some embodiments, the one or more cargo molecules is delivered with temporal and spatial control.

Some embodiments include a method for drug delivery comprising: applying focused ultrasound to an aqueous environment at least containing a concentration of at least one polymer and a plurality of gas-filled structures; wherein each of the at least one polymer comprises a polymer chain functionalized with at least one mechanophore, and wherein the at least one mechanophore comprises at least one drug molecule; and wherein the focused ultrasound is applied such that the plurality of gas-filled structures collapses thereby transducing the focused ultrasound to a mechanical force, such that the mechanical force activates the at least one mechanophore to release the at least one drug molecule into the aqueous environment.

In some embodiments, the at least one drug molecule is selected from the group consisting of: an anticancer drug, a chemotherapeutic drug, a small molecule drug, a biologic drug, a macromolecule drug, and a micromolecule drug.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention. It should be noted that the patent or application file contains at least one drawing executed in color. Copies of this patent or patent application publication with color drawing(s) will be provided by the Office upon request and payment of the necessary fee.

FIG. 1A illustrates ultrasound-induced mechanochemical activation of a mechanophore-functionalized polymer in solution in accordance with prior art.

FIG. 1B illustrates a schematic of activation of mechanochemical reactions using focused ultrasound enabled by gas-filled structures in accordance with an embodiment.

FIG. 1C illustrates a schematic of activation of mechanochemical reactions under physiological conditions using biocompatible focused ultrasound enabled by gas-filled structures as acousto-mechanical transducers in accordance with an embodiment.

FIGS. 2A through 2C illustrate analysis of biocompatible focused ultrasound conditions for mechanochemical activation in accordance with an embodiment.

FIG. 3 illustrates temperature increment profiles under focused ultrasound conditions that can lead to dangerous levels of heating in accordance with an embodiment.

FIGS. 4A through 4C illustrate mechanochemically mediated release of an aminocoumarin small molecule payload triggered using biocompatible focused ultrasound in accordance with an embodiment.

FIG. 5 illustrates release of aminocoumarin as a function of FUS exposure time in the presence and absence of GVs in accordance with an embodiment.

FIG. 6 illustrates a control experiment showing lack of release of aminocoumarin for the chain-end functionalized polymer in accordance with an embodiment.

FIGS. 7A through 7C illustrate mechanochemically triggered release of camptothecin using biocompatible focused ultrasound in accordance with an embodiment.

FIGS. 8A through 8E illustrate analysis of the thermally activated release of camptothecin from the mechanophore bis-initiator in accordance with an embodiment.

FIGS. 9A and 9B illustrate analysis of thermally activated mechanophore bis-initiator in accordance with an embodiment.

FIG. 10 illustrates no release of camptothecin in solution without ultrasound in accordance with an embodiment.

FIG. 11 illustrates the chemical structures of various compounds in accordance with an embodiment.

FIG. 12 illustrates construction of a calibration to determine the concentration of aminocoumarin in accordance with an embodiment.

FIG. 13 illustrates a calibration curve to determine the concentration of aminocoumarin in accordance with an embodiment.

FIG. 14 illustrates a calibration curve to determine the concentration of aminocoumarin in accordance with an embodiment.

FIG. 15 illustrates the release of aminocoumarin as measured by photoluminescence in accordance with an embodiment.

FIG. 16 illustrates the release of aminocoumarin as a function of sonication time in accordance with an embodiment.

FIG. 17 illustrates the release of aminocoumarin in water at about 50° C. as a function of incubation time in accordance with an embodiment.

FIG. 18 illustrates the release of aminocoumarin as a function of peak negative pressure with focused ultrasound in the presence and absence of gas vesicles in accordance with an embodiment.

FIG. 19 illustrates the release of aminocoumarin as a function of GV concentration in accordance with an embodiment.

FIG. 20 illustrates the release of aminocoumarin under focused ultrasound conditions with and without intact GVs in accordance with an embodiment.

FIG. 21 illustrates the release of aminocoumarin at various polymer concentrations using focused ultrasound with and without GVs in accordance with an embodiment.

FIG. 22 illustrates the relative response factor of camptothecin and internal standard for HPLC quantification in accordance with an embodiment.

FIG. 23 illustrates HPLC chromatograms confirming selective cargo release in the presence of GVs using biocompatible focused ultrasound in accordance with an embodiment.

FIG. 24 illustrates HPLC chromatograms for control experiments in accordance with an embodiment.

FIG. 25 illustrates HPLC chromatograms demonstrating stability of camptothecin under focused ultrasound conditions in accordance with an embodiment.

### DETAILED DESCRIPTION OF THE INVENTION

External control of specific chemical reactions with spatial and temporal precision can have great benefits for biomedical applications. The targeted delivery of therapeutic compounds to precise locations in the body can maximize accumulation of drugs at the sites of disease and reduce undesirable side effects to healthy tissues. Spatially triggered reactions can establish patterned engineered tissues and functional living materials. Among various ways to drive chemical transformations remotely, light is a prototypical external stimulus for achieving high spatiotemporal resolution, but its limited tissue penetration depth may hamper biological utility. (See, e.g., Y. Tao, et al., Adv. Funct. Mater. 30, 2005029 (2020); T. L. Rapp, et al., Adv. Drug Deliv. Rev. 171, 94-107 (2021); H. Kobayashi, et al., Chem. Rev. 110, 2620-2640 (2010); the disclosures of which are incorporated by reference.) Research in polymer mechanochemistry has focused on the development of force sensitive molecules termed mechanophores, including those that release covalently bound small molecules upon mechanochemical activation. (See, e.g., J. Li, C. Nagamani, et al., Acc. Chem. Res. 48, 2181-2190 (2015); B. A. Versaw, et al., J. Am. Chem. Soc. 143, 21461-21473 (2021); M. A. Ghanem, et al., Nat. Rev. Mater. 6, 84-98 (2021); the disclosures of which are incorporated by reference.) Mechanical force, which can be applied with spatial and temporal precision, is transduced to the mechanophore through covalently linked polymer chains to elicit a chemoselective response. Mechanophores have been developed to release carbon monoxide, acid, catalysts, and a variety of other payloads. (See, e.g., Y. Sun, et al., J. Am. Chem. Soc. 144, 1125-1129 (2022); S. Nijem, et al., Polym. Chem. 13, 3986-3990 (2022); C. E. Diesendruck, et al., J. Am. Chem. Soc. 134, 12446-12449 (2012); Y. Lin, et al., J. Am. Chem. Soc. 142, 99-103 (2020); H. Shen, et al., Angew. Chem. Int. Ed. 60, 13559-13563 (2021); the disclosures of which are incorporated by reference.) Previous research has shown general and modular mechanophore platforms enabling the mechanically triggered release of functionally diverse molecules that are especially promising for biomedical applications. (See, e.g., Z. Shi, et al., Chem. Sci. 12, 1668-1674 (2021); S. Huo, et al., Nat. Chem. 13, 131-139 (2021); X. Hu, et al., J. Am. Chem. Soc. 141, 15018-15023 (2019); X. Hu, et al., ACS Cent. Sci. 7, 1216-1224 (2021); T. Zeng, et al., Chem. Commun. 57, 11173-11176 (2021); U.S. patent application Ser. No. 17/469,728 to Robb et al., filed Sep. 8, 2021; the disclosures of which are incorporated by reference.)

Ultrasonication is an effective technique for achieving mechanophore activation in the laboratory. High intensity low frequency sonication (typically 20 kHz) using an immersion probe is commonly employed to exert mechanical forces on polymers in solution via acoustic cavitation, a process in which the nucleation, growth, and collapse of gas bubbles produces solvodynamic shear and results in the rapid elongation of polymer chains. (See, e.g., J. Li, et al., Acc. Chem. Res. 48, 2181-2190 (2015); P. A. May, et al., Chem. Soc. Rev 42, 7497-7506 (2013); the disclosures of which are incorporated by reference). However, the cavitation of dissolved gases under these conditions is highly destructive to tissues, making it incompatible with most biological applications. (See, e.g., B. A. Versaw, et al., J. Am. Chem. Soc. 143, 21461-21473 (2021); S. Mitragotri, et al., Nat. Rev. Drug Discov. 4, 255-260 (2005); the disclosures of which are incorporated by reference). Researches have used clinically relevant focused ultrasound (FUS) operating at higher frequencies for mechanophore activation in polymer networks and hydrogels. (See, e.g., G. Kim, et al., Proc. Natl. Acad. Sci. USA 116, 10214-10222 (2019); G. Kim, et al., Proc. Natl. Acad. Sci. USA 119, e2109791119 (2022); the disclosures of which are incorporated by reference.) Focused ultrasound is an external stimulus that can be applied with sub-millimeter spatial resolution and is highly penetrant to biological tissues. (See, e.g., W. J. Elias, et al., N. Engl. J. Med. 375, 730-739 (2016); the disclosure of which is incorporated by reference.) Nevertheless, while these earlier studies provide a promising direction, the high acoustic intensities employed can cause unsafe heating, which represents a major obstacle for biomedical applications. (See, e.g., B. Fowlkes, et al., Acoust. Today (2012) https:/doi.org/10.1121/1.4788649 (Jun. 6, 2023); the disclosure of which is incorporated by reference.) Moreover, coupling biocompatible ultrasound with the remote activation of mechanochemical reactions in solution remains an unsolved challenge. New methodologies are therefore required to realize the translational potential of polymer mechanochemistry.

### Ultrasound-Controlled Chemical Reactions

Many embodiments provide systems and methods for remote control of mechanochemical reactions in aqueous environments using biocompatible focused ultrasound. Several embodiments implement gas-filled structures as acousto-mechanical transducers that can be selectively collapsed with biocompatible focused ultrasound to activate various types of mechanophores to achieve the desired functional responses. In some embodiments, the gas-filled structures can selectively trigger the release of a variety of cargo molecules from mechanophore-functionalized polymers. Some embodiments implement gas vesicles as acousto-mechanical transducers that can be triggered by biocompatible focused ultrasound to facilitate the selective release of fluorogenic and therapeutic cargo molecules from mechanophore-functionalized polymers. Many embodiments can achieve remote control of the desired mechanochemical reactions with spatial and temporal precision in biologically relevant environments.

Many embodiments activate various types of mechanophores using ultrasound and gas-filled structures. In several embodiments, mechanophores can functionalize various types of polymer chains. The mechanophores can be attached to the polymer chains via chemical bonds such as (but not limited to) covalent bonds, bonds formed via bioconjugation, bonds formed via click chemistry. Ultrasound can apply mechanical forces that are needed to activate the chemical transformations of the mechanophores to achieve the desired applications. Some embodiments use ultrasound to activate mechanochromic mechanophores such as (but not limited to) spiropyran, derivatives of spiropyran, naphthopyran, benzopyran, rhodamine, oxazine, triarylmethane, and/or diarylbibenzofuranone to initiate the color changes and/or luminescence changes of the mechanophores. Some embodiments use ultrasound to activate mechanophores that can change electrical conductivity and/or reveal reactive groups. Examples of the mechanophores include (but are not limited to) ladderenes, derivatives of cyclobutane, benzocyclobutene, beta-lactams, dihalocyclopropranes, and/or Diels-Alder adducts.

Many embodiments implement mechanophore functionalized polymers to carry the desired cargo molecules. Mechanophores are chemical moieties that are sensitive to mechanical forces. In several embodiments, a variety of cargo molecules can be attached to the mechanophores. When triggered by mechanical forces, mechanophores can react selectively and release the cargo molecules. The cargo molecules can be attached to the mechanophore via chemical bonds such as (but not limited to) covalent bonds, bonds formed via bioconjugation, bonds formed via click chemistry. As can be readily appreciated, any of a variety of chemical bonds can be utilized as appropriate to the requirements of specific applications in accordance with various embodiments of the invention. In some embodiments, the polymer chains can be functionalized with different types of mechanophores. Some embodiments use mechanophores derived from 2-furylcarbinol derivatives for mechanically triggered molecular release. As can be readily appreciated, any of a variety of mechanophores can be utilized as appropriate to the requirements of specific applications in accordance with various embodiments of the invention.

In some embodiments, various types of cargo molecules can be attached to the mechanophores. The cargo molecules can be selected in accordance with the desired applications. The triggered release of the cargo molecules in accordance with many embodiments enables diverse biomedical applications such as (but not limited to) diagnostics, detection, sensing, therapeutics, imaging, and/or pharmaceuticals. The applications can be in situ, in vitro and/or in vivo for human and non-human subjects. Examples of the cargo molecules include (but are not limited to) macromolecules, small molecules, biomolecules, organic compounds, inorganic compounds, organometallic compounds, amino acids, polypeptides, proteins, monosaccharides, polysaccharides, nucleic acids, DNAs, RNAs, and any combinations thereof. The cargo molecules can have various functions such as (but not limited to) pharmaceutical molecules, drugs, chemotherapeutic drugs, biological drugs, catalysts, fluorescent molecules, fluorescent probes, fluorogenic probes, fluorophores, luminophores, molecules that can change color upon release, and any combinations thereof. In certain embodiments, the cargo molecules can be anticancer drugs such as (but not limited to) camptothecin. As can be readily appreciated, any of a variety of cargo molecules can be utilized as appropriate to the requirements of specific applications in accordance with various embodiments of the invention.

Many embodiments provide methods of delivery for in vivo applications. Some embodiments provide administration routes that involve systemic co-injection of the gas-filled structures and functionalized polymer, relying on these synergistic components to accumulate at sufficient concentration. In some embodiments, the gas-filled structures and polymers can be colocalized through non-covalent interactions or covalent anchoring, which may further enhance the local concentration of the two components necessary to achieve mechanochemical transduction. In some embodiments, the gas-filled structures and functionalized polymer can be confined within a semipermeable implant, enabling temporal control of mechanophore activation using ultrasound.

In many embodiments, various types and sizes of polymer chains can be used. In several embodiments, the polymer chains are soluble in aqueous solutions. In some embodiments, the polymer chains functionalized with the mechanophores and the cargo molecules are soluble in aqueous solutions. In several embodiments, the polymer chains, the mechanophores, and/or the cargo molecules are biocompatible and non-toxic to the intended subjects. The polymer chains can have various chain lengths and molar mass. A longer polymer chain may enable a faster mechanophore reaction upon mechanical activation. Examples of types of polymer chains include (but are not limited to) polyacrylates including poly(2-(methylsulfinyl)ethyl acrylate) (PMSEA), poly(oligoethylene glycol acrylate)s, and poly(2-hydroxyethyl acrylate), polymethacrylates, polyacrylamides such as poly(N-isopropyl acrylamide), polyoxazolines, poly(ethylene glycol) (PEG), polypeptides, polyesters, and polysaccharides. In some embodiments, the mechanophores can be attached to a suitable position of the polymer chain such as (but not limited to) in the middle of a polymer chain, at an end of a polymer chain, or any position along a polymer chain.

Many embodiments apply a mechanical force using biocompatible ultrasound. In many embodiments, the mechanochemical activation reactions can take place at a temperature from about 5° C. to about 10° C.; or from about 10° C. to about 15° C.; or from about 15° C. to about 20° C.; from about 20° C. to about 25° C.; or from about 25° C. to about 30° C.; or from about 30° C. to about 35° C.; or from about 35° C. to about 40° C.; or from about 40° C. to about 45° C.; or from about 45° C. to about 50° C.; or greater than or equal to about 50° C. In some embodiment, the mechanical activation reactions can take place at room temperature (from about 20° C. to about 25° C.). In some embodiments, the mechanical activation reactions can take place at body temperature (from about 36° C. to about 38° C.). In some embodiments, the mechanical activation reactions can take place at an elevated temperature greater than or equal to about 25° C.

In many embodiments, the chemical reactions take place in solution. In some embodiments, the solution is an organic solution. In some embodiments, the solution is an aqueous environment. In several embodiments, the aqueous environment is the desired physiological environment for the mechanophores, the mechanophore functionalized polymers, and/or the intended subjects. In some embodiments, the aqueous environment is in water or a water-based solution. In some embodiments, the aqueous environment is in a buffer solution. In some embodiments, the aqueous environment is a cellular environment such as (but not limited to) an intercellular environment, and/or an intracellular environment. In some embodiments, the aqueous environment is an in situ environment, an in vitro environment, or an in vivo environment. In some embodiments, the aqueous environment is a physiological environment. In some embodiments, the aqueous environment is a clinically relevant environment.

In many embodiments, molecular release occurs selectively in the presence of gas-filled structures upon exposure to ultrasound. The gas-filled structures are pressure sensitive. In many embodiments, the gas-filled structures can function as acousto-mechanical transducers to effectively couple ultrasound operating under physiologically relevant conditions with the mechanochemical activation of mechanophore-containing polymers. The acousto-mechanical transducer, or acoustosensitizer, can transduce acoustic waves to mechanical energy. Mechanical energy activates the mechanophore functionalized polymers such that the mechanophores can achieve various functional responses such as (but not limited to) color changes, modulating electrical conductivity, sensing forces, revealing reactive groups, and/or releasing attached cargo molecules.

In many embodiments, gas-filled structures refer to gas-filled microstructures, gas-filled nanostructures, gas vesicles, natural gas vesicles, synthetic gas vesicles, and/or microbubbles, unless specified otherwise. Gas vesicles (GVs) are a type of genetically encodable, air-filled protein nanostructure that can be used as contrast agents for non-invasive biomedical imaging, including high-frequency diagnostic ultrasound. Microbubbles are small, gas-filled bubbles that can be used as contrast agents in medical imaging. In several embodiments, the gas-filled structures have a stable structure before being activated by the mechanical forces. In some embodiments, the gas-filled structures can have spherical shapes; or ellipsoid shapes; or cylindrical shapes; or polygon shapes; or cubical shapes; or cuboid shapes; or triangular prism shapes; or triangular pyramid shapes; or square pyramid shapes; or cone shapes; or tetrahedron shapes; or hexagon shapes; or any combinations thereof. In several embodiments, the gas-filled structures can enclose a non-reactive gas such as (but not limited to) air, nitrogen, carbon dioxide, an inert gas, helium, argon, neon, xenon, and a combination of any of the non-reactive gases. In several embodiments, the gas-filled structures can have an average diameter ranging from about 50 nm to about 10 microns; or from about 50 nm to about 100 nm; or from about 100 nm to about 200 nm; or from about 200 nm to about 300 nm; or from about 300 nm to about 400 nm; or from about 400 nm to about 500 nm; or from about 500 nm to about 600 nm; or from about 600 nm to about 700 nm; or from about 700 nm to about 800 nm; or from about 800 nm to about 900 nm; or from about 900 nm to about 1 micron; or from about 1 micron to about 5 microns; or from about 5 microns to about 10 microns. In several embodiments, the gas-filled structures can have an average length ranging from about 50 nm to about 10 microns; or from about 50 nm to about 100 nm; or from about 100 nm to about 200 nm; or from about 200 nm to about 300 nm; or from about 300 nm to about 400 nm; or from about 400 nm to about 500 nm; or from about 500 nm to about 600 nm; or from about 600 nm to about 700 nm; or from about 700 nm to about 800 nm; or from about 800 nm to about 900 nm; or from about 900 nm to about 1 micron; or from about 1 micron to about 5 microns; or from about 5 microns to about 10 microns. In some embodiments, the gas vesicles can have an average diameter from about 45 nm to about 250 nm and an average length from about 100 nm to about 600 nm. In certain embodiments, the gas vesicles can have amphiphilic protein shells that are gas-permeable but exclude liquid water from their hydrophobic interior. Gas vesicles can act as seeds for bubble formation and cavitation upon exposure to biocompatible ultrasound. Several embodiments apply biocompatible ultrasound pulses to the gas-filled structures. Under sustained ultrasound pulses with relatively low-pressure levels, large bubbles liberated from the rupture of the gas-filled structures can undergo rapid growth followed by intense collapse in an inertial cavitation process with pronounced mechanical effects. With the biocompatible ultrasound parameters in accordance with several embodiments, which are otherwise benign to tissues and/or cells, these mechanical effects take place only in the vicinity of gas-filled structures.

In many embodiments, biocompatible focused ultrasound conditions are applied to trigger the activation of mechanochemical reactions. Insonation can result in unsafe heating and tissue damage. Non-specific acoustic cavitation can cause undesirable thermal and/or mechanical effects and disrupt tissues. The biocompatible focused ultrasound conditions applied in conjunction with gas vesicles in accordance with several embodiments, are selected to minimize and/or eliminate overheating and tissue damage. In many embodiments, the biocompatible ultrasound conditions can cause an increase in the temperature of the medium of less than or equal to about 5° C. In several embodiments, an upper boundary of biocompatible ultrasound conditions can be the threshold at which acoustic cavitation can be activated selectively in the presence of gas vesicles. The upper boundary conditions in accordance with several embodiments do not include the conditions without the gas vesicles.

In some embodiments, the biocompatible ultrasound conditions can have an acoustic intensity Iₛₚₚₐ (spatial-peak-pulse-average) less than or equal to about 1000 W/cm². The acoustic intensity Iₛₚₚₐ can prevent mechanical damage to tissues. In some embodiments, the biocompatible ultrasound conditions can have an acoustic intensity Iₛₚₜₐ (spatial-peak-time-average) less than or equal to about 50 W/cm². The acoustic intensity Iₛₚₜₐ can effectively suppress thermal damage to tissues. In certain embodiments, Iₛₚₚₐ is less than or equal to about 1000 W/cm² and Iₛₚₜₐ is less than or equal to about 50 W/cm². In certain embodiments, Iₛₚₚₐ is less than or equal to about 900 W/cm² and Iₛₚₜₐ is less than or equal to about 45 W/cm². In certain embodiments, Iₛₚₚₐ is less than or equal to about 800 W/cm² and Iₛₚₜₐ is less than or equal to about 40 W/cm². In certain embodiments, Iₛₚₚₐ is less than or equal to about 700 W/cm² and Iₛₚₜₐ is less than or equal to about 35 W/cm². In certain embodiments, Iₛₚₚₐ is less than or equal to about 600 W/cm² and Iₛₚₜₐ is less than or equal to about 30 W/cm². In certain embodiments, Iₛₚₚₐ is less than or equal to about 500 W/cm² and Iₛₚₜₐ is less than or equal to about 25 W/cm². In certain embodiments, Iₛₚₚₐ is less than or equal to about 400 W/cm² and Iₛₚₜₐ is less than or equal to about 20 W/cm². In certain embodiments, Iₛₚₚₐ is less than or equal to about 300 W/cm² and Iₛₚₜₐ is less than or equal to about 15 W/cm². In certain embodiments, Iₛₚₚₐ is less than or equal to about 200 W/cm² and Iₛₚₜₐ is less than or equal to about 10 W/cm². In certain embodiments, Iₛₚₚₐ is less than or equal to about 100 W/cm² and Iₛₚₜₐ is less than or equal to about 5 W/cm². In certain embodiments, Iₛₚₚₐ is less than or equal to about 90 W/cm² and Iₛₚₜₐ is less than or equal to about 5 W/cm². In certain embodiments, Iₛₚₚₐ is less than or equal to about 80 W/cm² and Iₛₚₜₐ is less than or equal to about 4 W/cm².

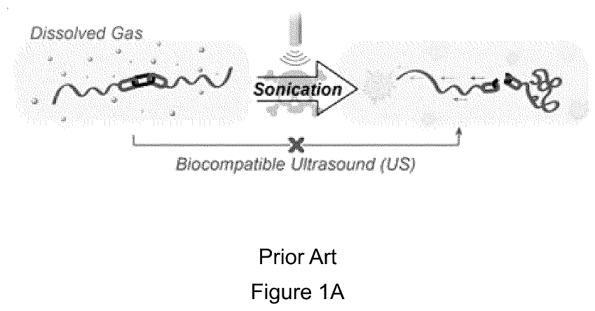

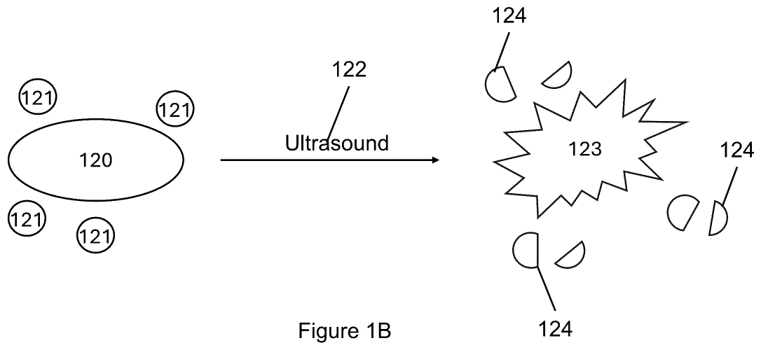

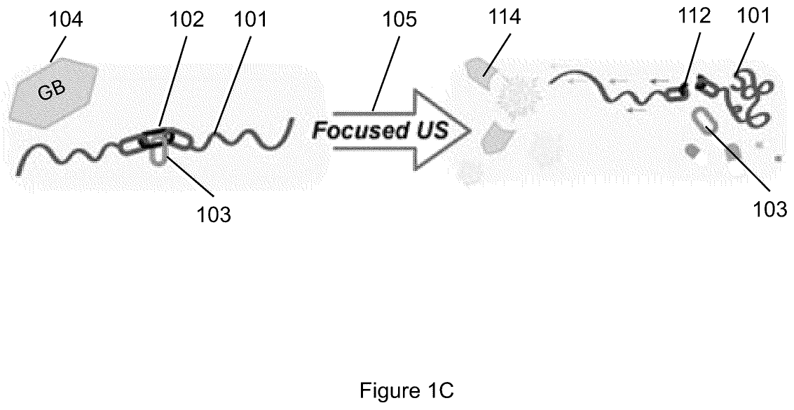

FIG. 1A illustrates ultrasound induced mechanochemical activation of a mechanophore-functionalized polymer in solution in accordance with prior art. Conventional methods of achieving ultrasound-induced mechanochemical activation rely on the cavitation of dissolved gases in solution using hazardous sonication at low frequency and high acoustic intensity. The hazardous sonication conditions can cause thermal and mechanical damage to tissue.

FIG. 1B illustrates a schematic of activation of mechanochemical reaction using biocompatible ultrasound under physiological conditions in accordance with an embodiment. Mechanophore modified polymers and/or mechanophores 120 can include various types of mechanophores. The mechanophores can be selected based on the desired applications. The mechanophore modified polymers and/or mechanophores 120 are soluble in organic or aqueous solutions at various temperatures. The mechanophores can have a variety of functional responses such as (but not limited to) releasing cargo molecules, changing colors, sensing forces, changing electrical conductivity, and/or changing structures to reveal functional groups. The mechanophore modified polymers and/or mechanophores 120 can be activated by applying ultrasound 122 in the presence of a plurality of gas-filled structures 121. Gas-filled structures 121 can be used to selectively activate the mechanochemical reactions. The gas-filled structures 121 can be filled with air or any type of unreactive gas. The gas-filled structures 121 can be air-filled protein nanostructures such as gas vesicles or microbubbles. The applied ultrasound 122 can be biocompatible and does not induce dangerous conditions for physiological environments. Focused ultrasound 122 can be applied to the solution where the gas-filled structures 121 and the mechanophore-functionalized polymer 120 are co-located. The focused ultrasound 122 triggers the gas-filled structures to collapse 124 and transduces mechanical forces that selectively activate the mechanophores 123 such that the functional responses of the mechanophores 123 are achieved.

FIG. 1C illustrates a schematic of a mechanochemical reaction activated under physiological conditions using biocompatible focused ultrasound enabled by gas-filled structures as acousto-mechanical transducers in accordance with an embodiment. Using gas-filled structures such as gas vesicles and/or microbubbles, which function as acousto-mechanical transducers, mechanochemical activation of mechanophore-functionalized polymers is achieved using biocompatible focused ultrasound under physiological conditions, enabling the controlled release of molecular cargo. A water-soluble polymer chain 101 can be functionalized with a mechanophore 102. The mechanophore 102 can be positioned at any suitable position of the polymer chain such that is experiences sufficient force during polymer chain extension. The mechanophore 102 can have a cargo molecule 103 conjugated to it. Gas-filled structures 104 can be used to selectively activate the mechanochemical reactions upon insonation. The gas-filled structures 104 can function as seeds for bubble formation and cavitation upon application of biocompatible ultrasound. The gas-filled structures 104 can be filled with air or any type of unreactive gas. The gas-filled structures 104 can be air-filled protein nanostructures such as gas vesicles. The gas-filled structures 104 can be microbubbles. Biocompatible focused ultrasound 105 can be applied to the fluid where the gas-filled structures 104 and the mechanophore-functionalized polymer are co-located. The biocompatible focused ultrasound 105 triggers the gas-filled structures 114 to collapse and expel their gas contents. Coalescence of gas-filled structures ultimately leads to inertial cavitation, which transduces mechanical forces that selectively activate the mechanophores such that the mechanophore releases its cargo molecules 112. The mechanochemical activation of the mechanophores can be remotely controlled with spatial and temporal precision due to the use of focused ultrasound.

### EXEMPLARY EMBODIMENTS

Although specific embodiments of systems and apparatuses are discussed in the following sections, it will be understood that these embodiments are provided as exemplary and are not intended to be limiting.

### Example 1: Remote Control of Mechanochemical Reactions Under Physiological Conditions Using Biocompatible Focused Ultrasound

Several embodiments identify focused ultrasound parameters for safe operation under physiological conditions with a particular emphasis on maintaining low-to-moderate pressure levels and avoiding temperature increases greater than about 6° C. to stay within a biomedically relevant regime. Focused ultrasound applied at high acoustic intensities can induce coagulative necrosis in tissues resulting from thermal disruption. Some embodiments use about 330 kHz focused ultrasound on either an 800 μL water sample in a sealed plastic microcentrifuge tube or a tissue-mimicking agarose gel inside a water tank and monitor peak negative pressure (PNP) and sample temperature using a hydrophone and internal thermocouple, respectively. An upper limit for PNP of 1.47 MPa can be established with a 4.5% duty cycle (about 3000 cycles per pulse with about 5 Hz pulse repetition frequency), which can result in a maximum temperature increase of about 3.6° C. in the agarose phantom (about 7.3° C. inside the plastic tube) that equilibrates in less than about 2 min. This biocompatible upper limit results in a spatial peak-temporal average acoustic intensity (Iₛₚₜₐ) of about 3.6 W/cm² that is within the safety limits established for the use of ultrasound in therapeutic applications. Similar ultrasound parameters have been successfully applied for therapeutic purposes to delicate tissues like the brain in both live animal studies and human clinical trials. Using similar focused ultrasound parameters as those previously employed for mechanophore activation at about 330 kHz and about 916 kHz result in an unsafe temperature increase exceeding about 27° C. above baseline due to the significantly higher acoustic intensity.

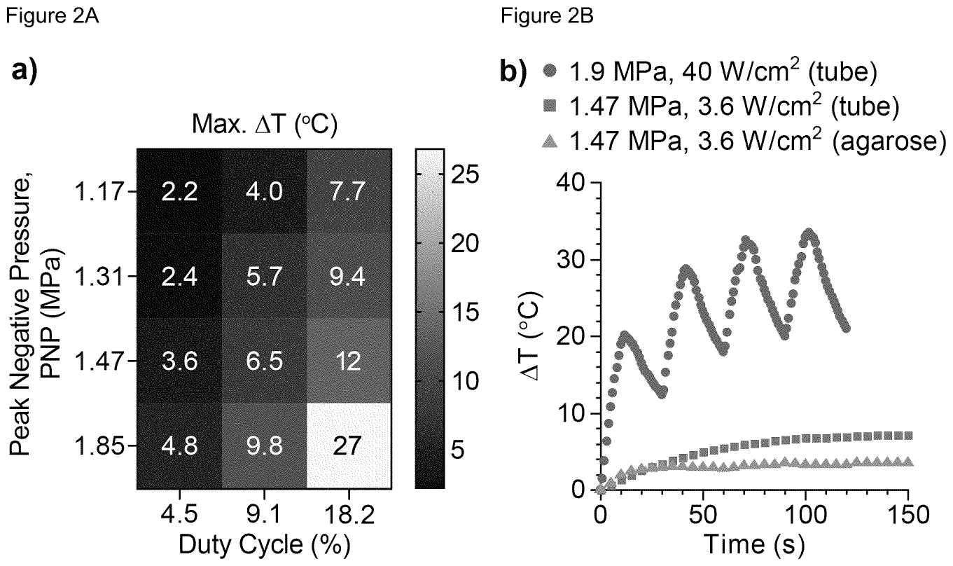

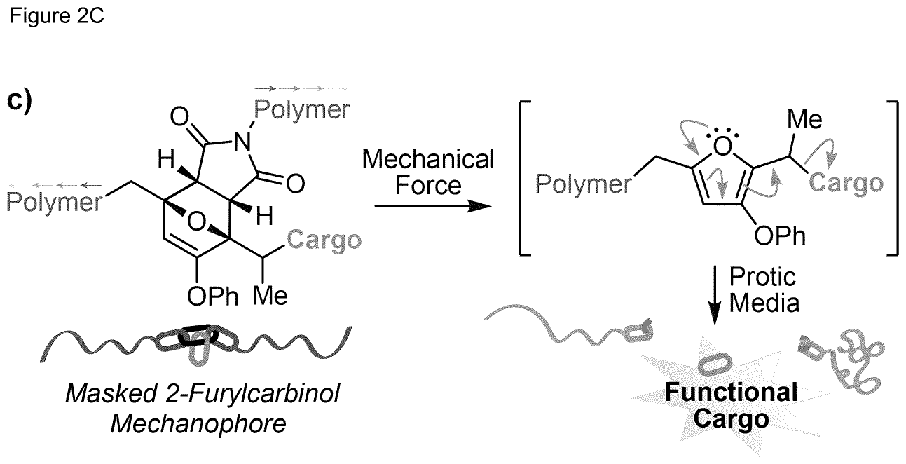

FIGS. 2A through 2C illustrate analysis of biocompatible focused ultrasound conditions for mechanochemical activation in accordance with an embodiment. FIG. 2A shows equilibrated maximum temperature increment (max. ΔT) at the focus of ultrasound in a tissue-mimicking 1% agarose hydrogel phantom with varying peak negative pressure and duty cycle. FIG. 2B shows the temperature profile of an 800 μL water sample at the focus of ultrasound inside a 2 mL plastic microcentrifuge tube or a tissue-mimicking agarose gel (phantom) with different ultrasound conditions (1.47 MPa PNP, 330 kHz, pulsed wave, 3000 cycles each pulse, 4.5% duty cycle, 3.6 W/cm² acoustic intensity, or 1.9 MPa PNP, 330 kHz, continuous wave, 10 s on/20 s off, 4 repeats, 40 W/cm² acoustic intensity).

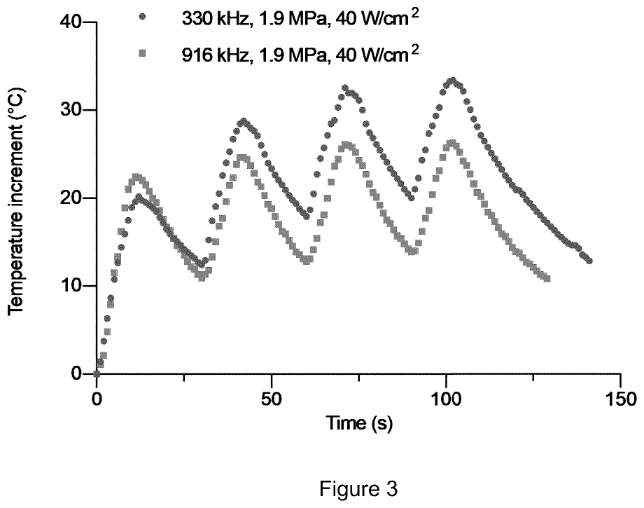

FIG. 3 illustrates temperature increment profiles under focused ultrasound conditions of 330 kHz and 916 kHz in accordance with an embodiment. Temperature increases above baseline of an 800 μL water sample inside a plastic microcentrifuge tube under 330 kHz or 916 kHz focused ultrasound. The temperature probe is inserted into the tube with the probe tip positioned at the tube center. Continuous-wave focused ultrasound is applied at 1.9 MPa peak negative pressure (PNP), 10 s on/20 s off, 4 repetitions, corresponding to a spatial peak-temporal average acoustic intensity (Iₛₚₜₐ) of 40 W/cm².

In several embodiments, mechanophore activation can be achieved under physiologically relevant conditions. In some embodiments, a masked 2-furylcarbinol mechanophore for mechanically triggered molecular release can be used. FIG. 2C shows a reaction scheme illustrating mechanically triggered molecular release from a masked 2-furylcarbinol mechanophore incorporated near the center of a polymer. Briefly, mechanochemical activation of the furan-maleimide Diels-Alder adduct reveals a latent 2-furylcarbinol derivative that spontaneously decomposes to release a covalently bound cargo molecule. Release occurs efficiently in polar protic environments and the molecular design is amenable to payloads conjugated to the mechanophore via a range of common functional groups including alcohols and amines. The incorporation of cargo molecules through covalent linkages increases resistance to nonspecific payload release in the absence of a specific triggering event compared to non-covalent encapsulation strategies, while the ability to precisely modulate the reactivity of mechanophores through structural modification using the tools of organic chemistry affords excellent selectivity and control over molecular release. Some embodiments implement water soluble, nontoxic polymer poly(2-(methylsulfinyl)ethyl acrylate) (PMSEA) that can be synthesized using controlled radical polymerization, enabling incorporation of the mechanophore near the middle of the polymer chain where force is maximized during solvodynamic extension. Alternative polymer conjugation strategies are also readily available, such as “click” coupling to further broaden the accessible materials.

Some embodiments use a PMSEA polymer containing a chain-centered mechanophore loaded with a fluorogenic aminocoumarin payload (PMSEA-CoumNH₂, Mₙ=260 kg/mol; Ð=1.47). Release of the aminocoumarin small molecule results in a pronounced increase in fluorescence that is easily detected and quantified spectroscopically. Exposing a dilute solution of the polymer (2 mg/mL) in water to 330 kHz focused ultrasound under the conditions identified above in the presence of gas vesicles (GVs, 1.4 nM) results in a strong fluorogenic response indicating the successful release of aminocoumarin. Approximately 15% release can be observed after 10 min of focused ultrasound exposure. Given their relative concentrations, about 800 equivalents of PMSEA-CoumNH₂ can be activated per GV within this timeframe. Extended exposure to focused ultrasound results in additional aminocoumarin release. Importantly, a fluorogenic response is not observed in the absence of GVs under the same conditions, confirming the GVs function as essential acousto-mechanical transducers, enabling mechanophore activation and selective cargo release under biocompatible conditions. Additional control experiments are performed on an analogous polymer with the mechanophore located at the chain end, which is not subjected to mechanical force. Minimal aminocoumarin release is observed under otherwise identical experimental conditions, confirming the mechanochemical origin of molecular release from PMSEA-CoumNH₂ upon exposure to focused ultrasound in the presence of GVs.

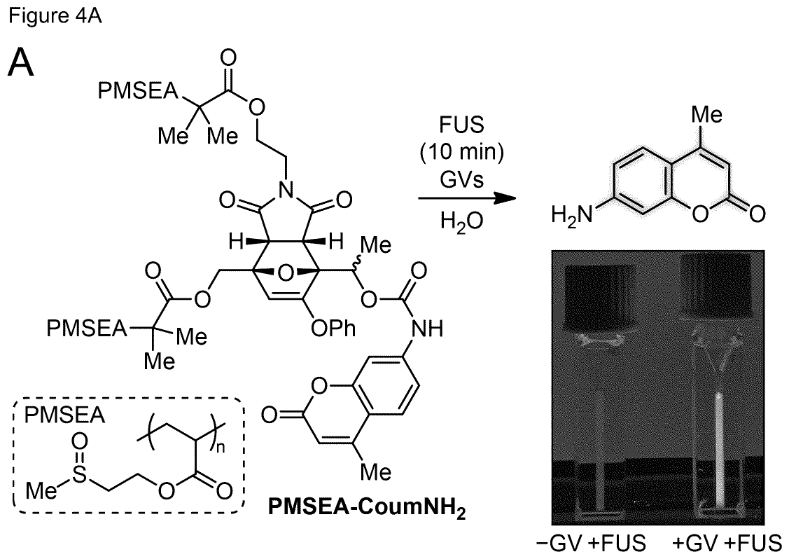

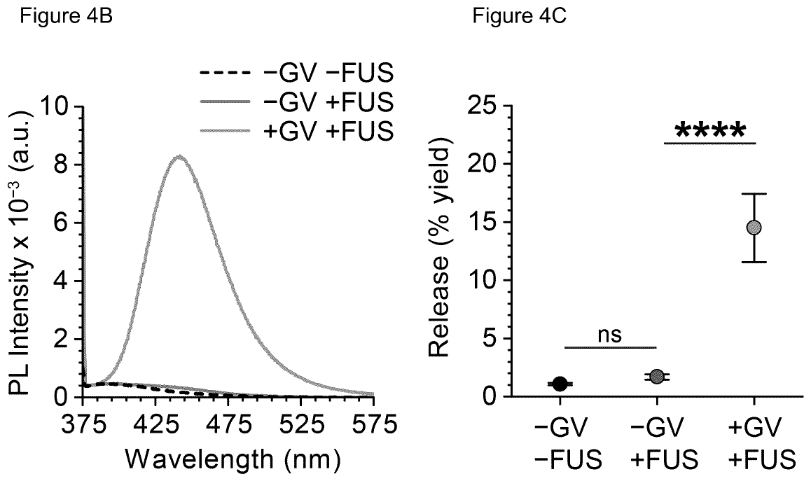

FIGS. 4A through 4C illustrate mechanochemically mediated release of an aminocoumarin small molecule payload triggered using biocompatible focused ultrasound in accordance with an embodiment. FIG. 4A shows biocompatible focused ultrasound (FUS, 330 kHz, 1.47 MPa PNP, 3.6 W/cm², 3000 cycles, 4.5% duty cycle, 10 min) triggers the release of aminocoumarin small molecule from PMSEA-CoumNH₂ (2 mg/mL in water) selectively in the presence of GVs (1.4 nM) resulting in a fluorogenic response. FIGS. 4B and 4C show the release of aminocoumarin characterized by photoluminescence (PL) spectroscopy. Error bars represent standard deviation from three replicate experiments for (−GV −FUS) and (−GV +FUS), and from four replicate experiments for (+GV +FUS). Asterisks represent statistical significance by ordinary one-way AVOVA with multiple comparison (****P<0.0001; ***P<0.001; **P<0.01; ns, not significant).

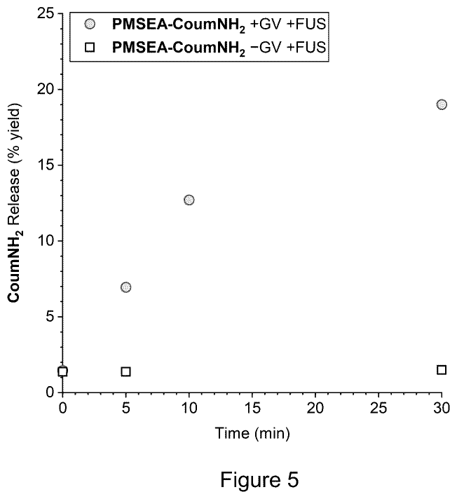

FIG. 5 illustrates release of aminocoumarin as a function of FUS insonation time in the presence and absence of GVs in accordance with an embodiment. FIG. 5 shows that the release of aminocoumarin (CoumNH₂) from PMSEA-CoumNH₂ (2 mg/mL, Mₙ=260 kg/mol, Ð=1.47) increases as a function of temporal exposure to biocompatible FUS (330 kHz, 3.6 W/cm², 1.47 MPa PNP, 4.5% duty cycle) in the presence of GVs (1.4 nM). Release of CoumNH₂ does not occur in the absence of GVs even after 30 min of insonation.

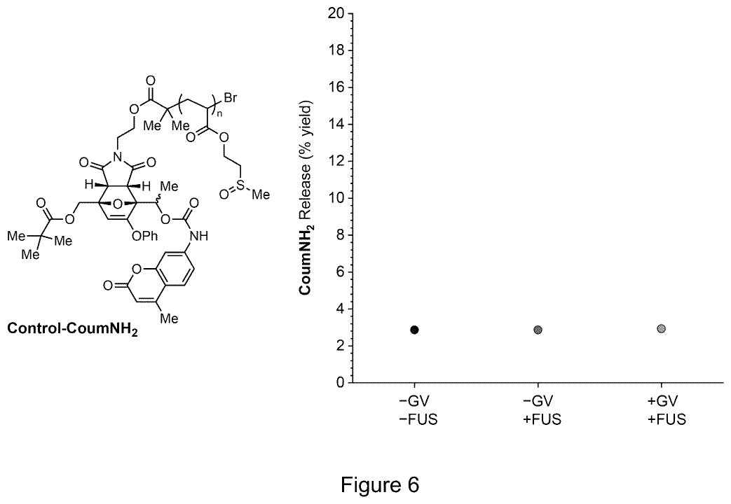

FIG. 6 illustrates insignificant release of aminocoumarin from chain-end functionalized polymer in accordance with an embodiment. FIG. 6 shows no significant increase in PL intensity is observed from chain-end functionalized polymer Control-CoumNH₂ (2 mg/mL, Mₙ=244 kg/mol, Ð=1.51) upon exposure to biocompatible FUS (330 kHz, 3.6 W/cm², 1.47 MPa PNP, 4.5% duty cycle, 10 min) in the absence of GVs.

Several embodiments investigate the mechanically triggered release of a small molecule therapeutic to demonstrate the capability of this system for drug delivery applications. Camptothecin can be used as a chemotherapeutic anticancer agent. Following a similar procedure as above, camptothecin can be conjugated to the mechanophore bis-initiator through a carbonate linkage followed by polymerization to afford PMSEA-CPT (Mₙ=319 kg/mol; Ð=1.47). Preliminary experiments performed on an isolated small molecule furfuryl carbonate model compound resembling the intermediate that is unmasked upon mechanophore activation confirm that camptothecin is successfully released in aqueous media, as evidenced by ¹H NMR spectroscopy and high-performance liquid chromatography (HPLC) measurements. Importantly, PMSEA-CPT is stable in aqueous solution, with negligible release of camptothecin detected over a period of 2 months under ambient conditions.

The triggered release of camptothecin from PMSEA-CPT is then investigated using biocompatible focused ultrasound in the presence of GVs under the physiologically relevant conditions established above. Approximately 8% release of camptothecin can be achieved after 10 min exposure to focused ultrasound in the presence of GVs, as evidenced by quantitative HPLC measurements. Similar to the results above for the release of aminocoumarin, no camptothecin release can be detected upon exposure of PMSEA-CPT to focused ultrasound in the absence of GVs. To assess the feasibility of this synergistic platform for targeted chemotherapy, cytotoxicity is investigated in vitro on the viability of Raji cells, a diffuse large B-cell lymphoma model of non-Hodgkin lymphomas, using an MTT colorimetric assay. Cells are treated with samples of PMSEA-CPT before and after mechanochemical activation with focused ultrasound at various concentrations and incubated for 2 days at 37° C. Cells incubated with PMSEA-CPT that was previously exposed to focused ultrasound in the presence of GVs exhibit a significant decrease in viability with a half-maximal effective concentration (EC₅₀) of about 250 nM. This EC₅₀ is approximately one order of magnitude higher than cells treated with isolated camptothecin small molecule, fully consistent with the extent of cargo release after 10 min of insonation characterized above. In contrast, no significant cytotoxicity is observed across all polymer concentrations in control experiments performed with PMSEA-CPT that is not exposed to focused ultrasound, with PMSEA-CPT that is exposed to ultrasound in the absence of GVs, or with a chain-end functional control polymer (PMSEA-Control) subjected to identical focused ultrasound conditions in the presence of GVs.

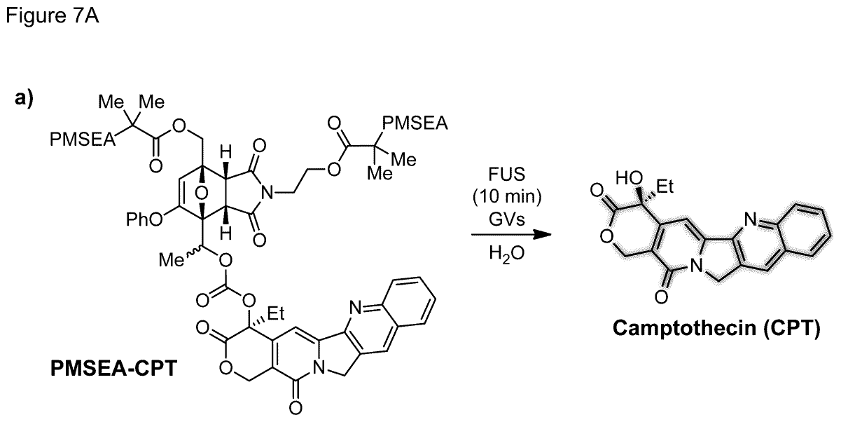

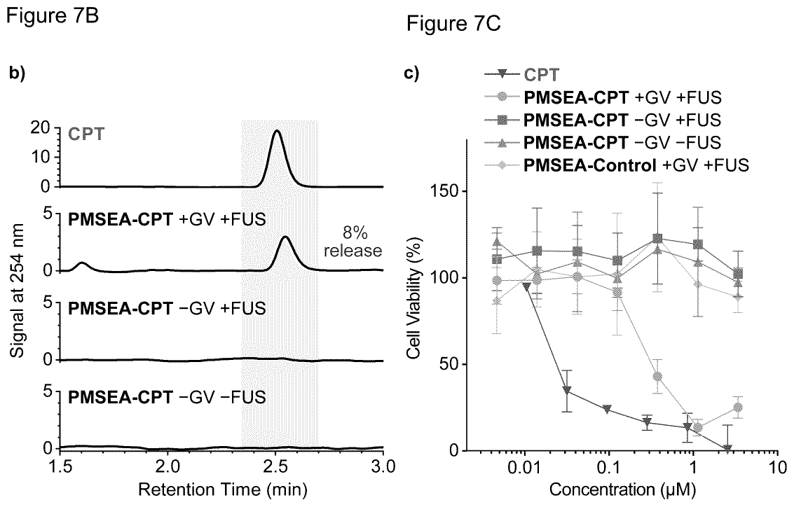

FIGS. 7A through 7C illustrate mechanochemically triggered release of camptothecin using biocompatible focused ultrasound in accordance with an embodiment. FIG. 7A shows biocompatible FUS (330 kHz, 1.47 MPa PNP, 3.6 W/cm², 3000 cycles, 4.5% duty cycle, 10 min) triggers the release of the small molecule topoisomerase inhibitor camptothecin (CPT) from PMSEA-CPT (2 mg/mL in water) selectively in the presence of GVs (1.4 nM). FIG. 7B shows characterization of CPT release using HPLC. Release of CPT occurs selectively upon exposure to FUS only in presence of GVs. FIG. 7C shows cell viability assays (MTT assay on Raji cell line, incubated 2 days at 37° C.) illustrating a significant decrease in viability for PMSEA-CPT exposed to FUS in the presence of GVs with a half-maximal effective concentration (EC₅₀) of about 250 nM. The EC₅₀ for pure CPT is about 25 nM, consistent with the extent of CPT release from PMSEA-CPT. Cell viability is not diminished in control experiments across the concentration range studied. Error bars represent standard deviation from three measurements each on samples from three replicate experiments.

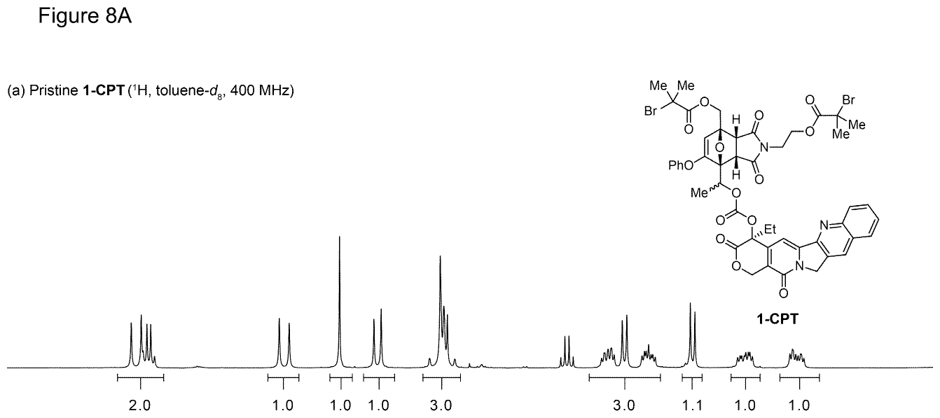

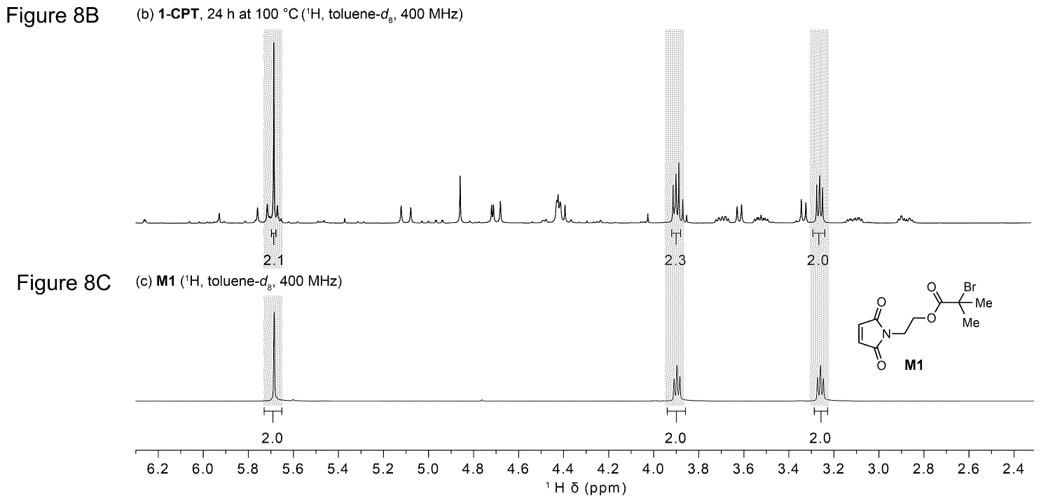

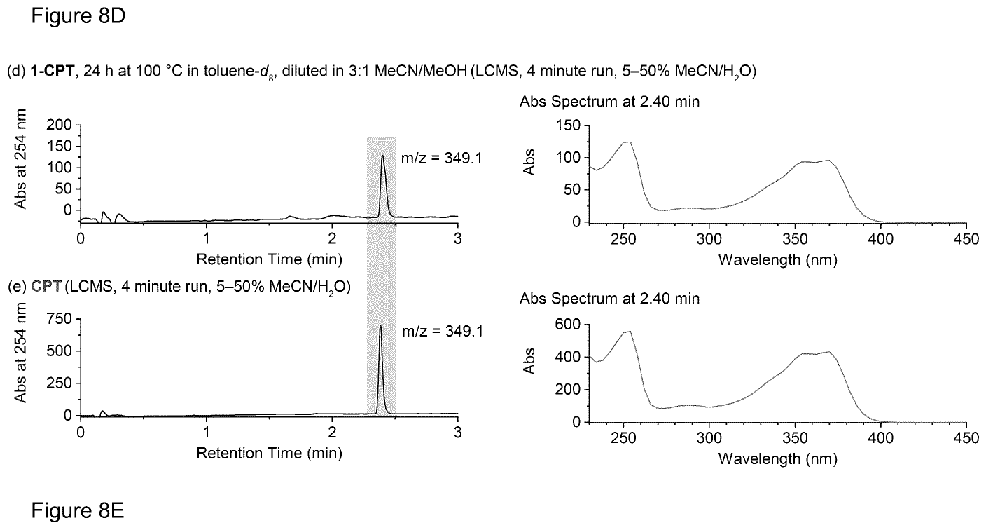

FIGS. 8A through 8E illustrate analysis of the thermally activated release of camptothecin from the mechanophore bis-initiator in accordance with an embodiment. The release of camptothecin (CPT) is observed following the thermally-induced retro-Diels-Alder reaction of 1-CPT and subsequent dilution in polar protic solvent. Comparison of partial ¹H NMR spectra of (FIG. 8A) pristine 1-CPT, (FIG. 8B) 1-CPT after heating for 24 h at 100° C., and (FIG. 8C) maleimide M1 indicates significant retro-Diels-Alder reaction occurs thermally. Subsequent analysis by LCMS with UV-vis absorption spectroscopy of (FIG. 8D) the NMR solution of 1-CPT after heating for 24 h at 100° C. diluted into 3:1 acetonitrile/methanol, and (FIG. 8E) pure CPT standard supports that CPT is released from thermally activated 1-CPT in polar protic solvent. Good agreement with the CPT standard is observed in retention time, mass-to-charge ratio, and absorption spectrum.

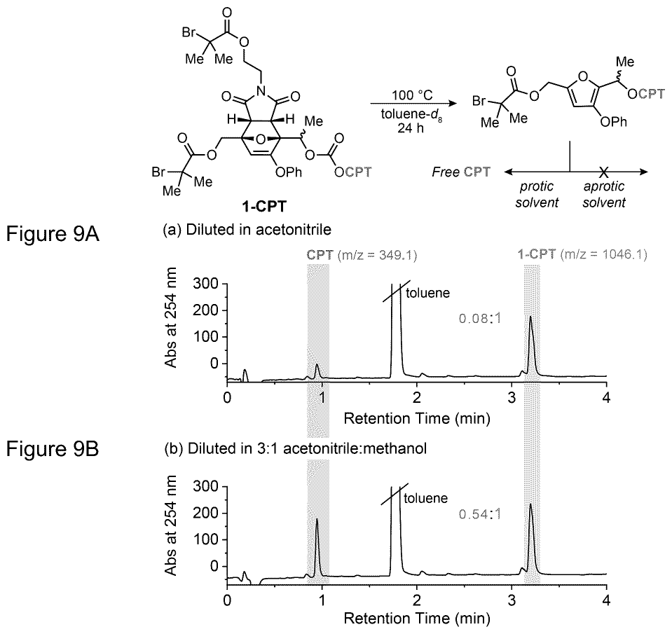

FIGS. 9A and 9B illustrate analysis of thermally activated mechanophore bis-initiator in accordance with an embodiment. The thermally activated 1-CPT (24 h at 100° C. in toluene-d₈) immediately after dilution in either (FIG. 9A) aprotic acetonitrile, or (FIG. 9B) 3:1 acetonitrile/methanol using LCMS with UV-vis absorption spectroscopy. The amount of CPT relative to remaining unreacted 1-CPT is significantly greater after dilution in protic solvent consistent with the expected reactivity of the furfuryl carbonate.

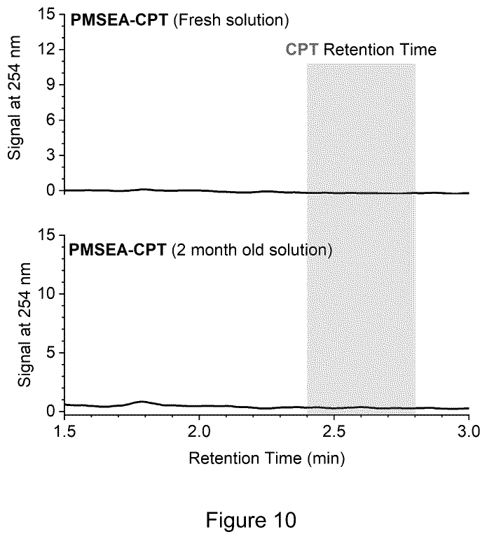

FIG. 10 illustrates no release of CPT without ultrasound in accordance with an embodiment. No release of camptothecin (CPT) from PMSEA-CPT (4 mg/mL, Mₙ=319 kg/mol, Ð=1.47) is observed in aqueous solution, even after 2 months in ambient conditions. The same sample volume was used for each injection.

Several embodiments provide a synergistic system that couples biocompatible focused ultrasound with the solution-phase activation of mechanochemical reactions under physiologically relevant conditions. Some embodiments use pressure-sensitive air-filled protein nanostructures called gas vesicles that function as acousto-mechanical transducers, effectively converting focused ultrasound energy into a coordinated mechanical stimulus for the activation of mechanophore-containing polymers. The mechanically triggered release of molecular payloads aminocoumarin and camptothecin from polymers in aqueous media illustrates the power of this approach for noninvasive bioimaging and therapeutic applications of polymer mechanochemistry. Several embodiments provide methods for remote control of specific chemical reactions under biomedically relevant conditions with the spatiotemporal precision and tissue penetration afforded by focused ultrasound.

### Example 2: Materials and Methods

Some embodiments provide the preparation methods of PMSEA-CoumNH₂ and PMSEA-CPT. The masked 2-furylcarbinol mechanophore with an aminocoumarin payload can be synthesized according to the literature. (See, e.g., X. Hu, et al., ACS Cent. Sci. 7, 1216-1224, 2021; the disclosure of which is incorporated by reference.) A similar procedure was used to synthesize the mechanophore incorporating camptothecin as the payload. The monomer 2-methylsulfinyl ethyl acrylate (MSEA) was prepared according to the literature. (See, e.g., S. Li, et al., Biomacromolecules 18, 475-482, 2017; the disclosure of which is incorporated by reference.) Water soluble linear PMSEA polymers incorporating the mechanophores were synthesized by controlled radical polymerization from the mechanophore initiator. Polymers were purified by precipitation into diethyl ether three times and subsequently dialyzed against deionized water for at least five cycles prior to use.

Some embodiments provide general protocols for FUS experiments. GVs were produced and purified from cyanobacteria Anabaena flos-aquae. Polymer solutions and GV suspensions were prepared using MilliQ water and separately placed under vacuum at 0.5 atm overnight to remove transiently trapped air bubbles prior to treatment with FUS. The GV suspension and polymer solution were gently mixed in a 2 mL microcentrifuge tube to achieve the desired concentrations of each component without introducing observable air bubbles. The total sample volume was 800 μL. The microcentrifuge tube was stabilized and exposed to FUS on a custom stage that positions the center of the sample at the focus of the FUS transducer operating at 330 kHz (fundamental frequency) or 916 kHz (third harmonic). After FUS exposure, samples were subsequently analyzed by fluorescence spectroscopy or high performance liquid chromatography, or evaluated for cytotoxicity.

Some embodiments provide cell viability assays procedures. Raji cells were incubated in RPMI 1640 media with 10% fetal bovine serum and 1% antibacterial Penicillin/Streptomycin (PenStrep) at 37° C. and 5% CO₂. An MTT colorimetric assay was used to quantify cell viability.

NMR spectra were recorded using a 400 MHz Bruker Avance III HD with Prodigy Cryoprobe. All ¹H NMR spectra are reported in δ units, parts per million (ppm), and were measured relative to the signals for residual chloroform (7.26 ppm) or toluene (2.08 ppm) in deuterated solvent. All ¹³C NMR spectra were measured in deuterated solvents and are reported in ppm relative to the signals for chloroform (77.16 ppm). Multiplicity and qualifier abbreviations are as follows: s=singlet, d=doublet, t=triplet, q=quartet, dd=doublet of doublets, dq=doublet of quartets, ABq=AB quartet, m=multiplet, br=broad.

High resolution mass spectra (HRMS) were obtained via direct injection on an Agilent 1260 Infinity II Series HPLC coupled to a 6230 LC/TOF system in electrospray ionization (ESI+) mode.

Polymer molar mass and dispersity was determined on a gel permeation chromatography (GPC) system with an Agilent pump (G7110B), equipped with an autosampler (G7129A), a Wyatt DAWN 8 multi-angle laser light scattering detector (λ=658.9 nm), a Wyatt Optilab refractive index detector (RI) (λ=658 nm), and an Agilent PL Aquagel-OH MIXED-H column. Aqueous buffer was prepared containing 0.2 M NaNO₃ with 200 ppm NaN₃. Filtered aqueous buffer was used as the eluent at a flow rate of 0.3 mL/min at 25° C. The data were analyzed using Wyatt Astra 7 with dn/dc=0.153 mL/g to obtain average molar masses (M_{w} and Mₙ) for each PMSEA polymer reported.

Photoluminescence spectra were recorded on a Shimadzu RF-6000 spectrofluorophotometer using a quartz microcuvette (Starna Cells 18F-Q-10-GL14-C, 10×2 mm). Excitation and emission slit widths were 5 nm and 3 nm, respectively. A Tecan Spark microplate reader was also used to acquire photoluminescence spectra in some experiments with 96-well microplate. Excitation and emission slit widths were both 5 nm.

High-Performance Liquid Chromatography (HPLC) measurements were performed with an Agilent Eclipse Plus C18 column or a C8 column using an in-line UV-vis detector and acetonitrile/water as the eluent.

Sonication experiments with an immersion probe were performed using a 500 Watt Vibra Cell 505 liquid processor (20 kHz) equipped with a 0.5-inch diameter solid probe, sonochemical adapter, and a Suslick reaction vessel.

LCMS measurements were performed with an Agilent 6140 Series Quadrupole LCMS Spectrometer System equipped with an Agilent Eclipse Plus C18 column using an in-line UV-vis detector and acetonitrile/water as the eluent.

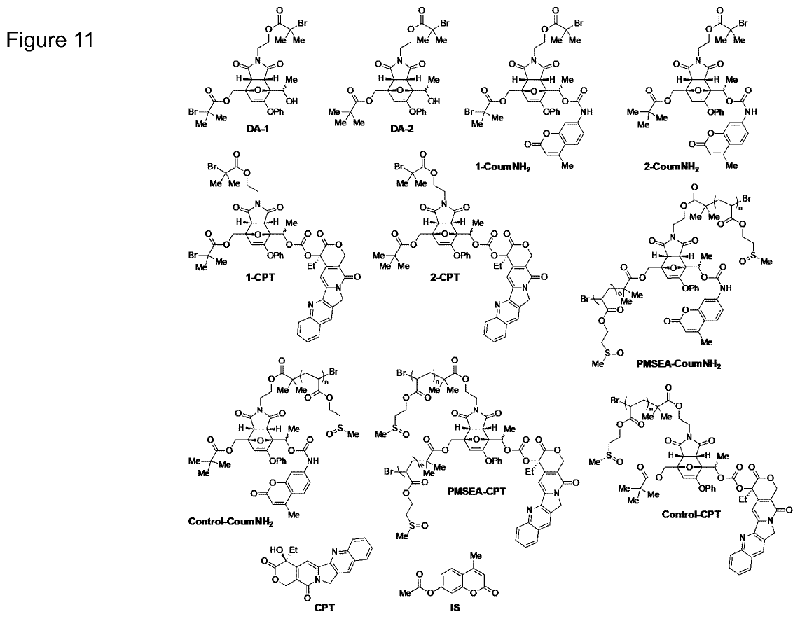

Compounds DA-1, DA-2, 1-CoumNH₂, and 2-CoumNH₂ were synthesized as reported previously (1). Internal standard 4-methyl-2-oxo-2H-chromen-7-yl acetate (IS) was prepared according to the literature (2). Monomer 2-methylsulfinyl ethyl acrylate (MSEA) was prepared according to the literature (3). FIG. 11 illustrates the chemical structures of various compounds in accordance with an embodiment.

Polymer solutions were dialyzed against deionized water for at least 5 cycles to remove any residual small molecule in the reaction mixture to prevent any aminocoumarin (CoumNH₂) or camptothecin (CPT) from affecting functional assays.

GVs were produced and purified from cyanobacteria Anabaena flos-aquae. GvpC was removed from the GVs by urea treatment followed by dialysis process. As a quality assurance step to confirm successful GvpC removal, pressure-dependent optical density (OD) measurements were performed for each sample at 0-7 bar to match previously reported measurements using an echoVis Vis-NIR light source coupled with an STS-VIS spectrometer and a 176.700-QS sample chamber. GV concentrations were measured as OD at 500 nm using a spectrophotometer. OD₅₀₀=1 corresponds to 114 pM of GV particles (4, 5).

Both polymer solution and GV suspension were placed in a vacuum environment at 0.5 atm overnight to remove transiently trapped air bubbles before FUS treatment. GV suspension and polymer solution were gently mixed to desired concentration without introducing observable air bubbles in a 2 mL microcentrifuge tube. The total volume of sample was 800 μL. The microcentrifuge tube was stabilized on a homemade holder which positions the center of the sample to be at the focus of a focused ultrasound transducer operating at 330 kHz (fundamental frequency) or 916 kHz (third harmonic).

### Example 3: Synthesis of Chemical Compounds

Synthesis of ((3aR,4R,7S,7aS)-2-(2-((2-bromo-2-methylpropanoyl)oxy)ethyl)-7-(1-(((((S)-4-ethyl-3,14-dioxo-3,4,12,14-tetrahydro-1H-pyrano[3′,4′:6,7]indolizino[1,2-b]quinolin-4-yl)oxy)carbonyl)oxy)ethyl)-1,3-dioxo-6-phenoxy-1,2,3,3a,7,7a-hexahydro-4H-4,7-epoxyisoindol-4-yl)methyl 2-bromo-2-methylpropanoatebromo-2-methylpropanoate (1-CPT). A flame-dried 25 mL round bottom flask was charged with 4-dimethylaminopyridine (DMAP) (544 mg, 4.43 mmol) and camptothecin (302 mg, 0.867 mmol). The flask was evacuated and refilled with N₂ three times, after which 5 mL of dichloromethane was added. Phosgene (15 wt % in toluene, 700 μL, 0.981 mmol) was added to the off-yellow suspension and the reaction was allowed to stir at room temperature. After 1 h the reaction had become homogenous and dark red, and DA-1 was added as a solution in 5 mL dichloromethane. The reaction continued to stir for 20 h, after which it was quenched with sat. NH₄Cl (8 mL). The products were extracted into dichloromethane (3×5 mL) and the combined organic layers were washed with brine (5 mL), dried with sodium sulfate, and filtered. The crude mixture was purified via silica gel chromatography (0-10% methanol/dichloromethane), followed by separation of the diastereomers by reverse-phase HPLC (70% acetonitrile/water). The title product (single diastereomer) was isolated as a light yellow solid (84 mg, 27%).

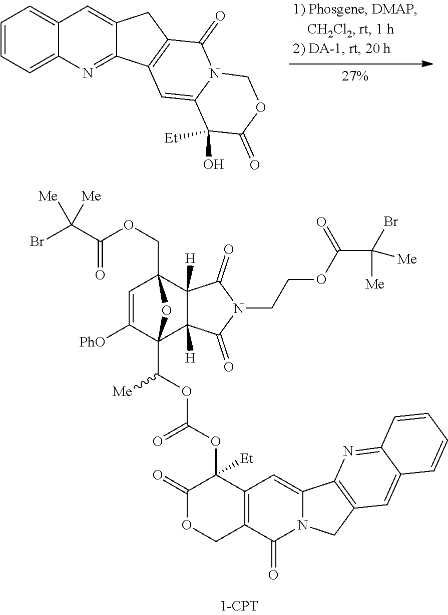 [SMILES: CC[C@@]1(O)C(=O)OCn2c1cc1c(c2=O)Cc2cc3ccccc3nc2-1 ; CC[C@@]1(OC(=O)OC(C)[C@]23O[C@](COC(=O)C(C)(C)Br)(C=C2Oc2ccccc2)[C@@H]2C(=O)N(CCOC(=O)C(C)(C)Br)C(=O)[C@@H]23)C(=O)OCc2c1cc1n(c2=O)Cc2cc3ccccc3nc2-1]

TLC (5% methanol/dichloromethane): R_{f}=0.55

¹H NMR (400 MHz, CDCl₃) δ: 8.38 (s, 1H), 8.07 (d, J=8.5 Hz, 1H), 7.91 (d, J=8.1 Hz, 1H), 7.84-7.76 (m, 1H), 7.68-7.59 (m, 1H), 7.36 (s, 1H), 7.25-7.18 (m, 2H), 7.13-7.05 (m, 1H), 6.90-6.81 (m, 2H), 5.69 (d, J=17.3 Hz, 1H), 5.49 (q, J=6.5 Hz, 1H), 5.39 (d, J=17.2 Hz, 1H), 5.26 (s, 2H), 4.98 (s, 1H), 4.66 (Abq, ΔV_{AB}=104 Hz, J_{AB}=12.5 Hz, 2H), 3.91-3.79 (m, 2H), 3.76-3.72 (m, 2H), 3.25-3.14 (m, 1H), 3.05-2.94 (m, 1H), 2.33 (dq, J=14.9, 7.4 Hz, 1H), 2.18 (dq, J=14.7 Hz, 7.5 Hz, 1H), 1.94 (s, 3H), 1.91 (s, 3H), 1.70 (s, 3H), 1.68 (s, 3H), 1.53 (d, J=6.6 Hz, 3H), 1.01 (t, J=7.4 Hz, 3H).

¹³C{¹H} NMR (101 MHz, CDCl₃) δ: 173.5, 172.6, 171.2, 171.1, 167.3, 162.2, 157.3, 154.4, 152.9, 152.4, 148.6, 146.5, 145.5, 131.4, 131.0, 130.0, 129.3, 128.7, 128.4, 128.23, 128.19, 125.9, 120.6, 119.4, 101.2, 95.8, 90.2, 88.3, 78.2, 71.8, 67.3, 63.6, 62.2, 55.6, 55.5, 51.4, 50.1, 47.8, 36.9, 32.0, 30.75, 30.71, 30.5, 30.4, 15.7, 7.7.

HRMS (ESI, m/z): calcd for [C₄₈H₄₆Br₂N₃O₁₄⁺] (M+H)⁺1046.1341, found 1046.1355.

Synthesis of ((3aR,4R,7S,7aS)-2-(2-((2-bromo-2-methylpropanoyl)oxy)ethyl)-7-(1-(((((S)-4-ethyl-3,14-dioxo-3,4,12,14-tetrahydro-1H-pyrano[3′,4′:6,7]indolizino[1,2-b]quinolin-4-yl)oxy)carbonyl)oxy)ethyl)-1,3-dioxo-6-phenoxy-1,2,3,3a,7,7a-hexahydro-4H-4,7-epoxyisoindol-4-yl)methyl pivalate (2-CPT). A flame-dried 25 mL round bottom flask was charged with DMAP (336 mg, 2.75 mmol) and camptothecin (192 mg, 0.552 mmol). The flask was evacuated and refilled with N₂ three times, after which 5 mL of dichloromethane was added. Phosgene (15 wt % in toluene, 430 μL, 0.602 mmol) was added to the off-yellow suspension and the reaction was allowed to stir at room temperature. After 1 h the reaction had become homogenous and dark red, and DA-2 was added as a solution in 5 mL of dichloromethane. The reaction continued to stir for 18 h, after which it was quenched with sat. NH₄Cl (8 mL). The products were extracted into dichloromethane (3×5 mL) and the combined organic layers were washed with brine (5 mL), dried with Na₂SO₄, and filtered. The crude mixture was purified via silica gel chromatography (0-10% methanol/dichloromethane), followed by separation of the diastereomers by reverse-phase HPLC (70% acetonitrile/water). The title product (single diastereomer) was isolated as a light yellow solid (50 mg, 28%).

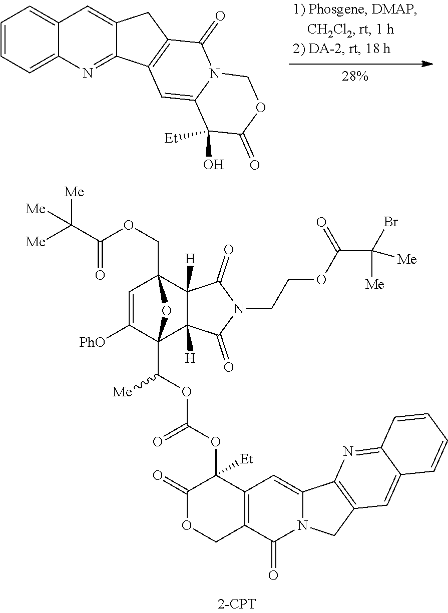 [SMILES: CC[C@@]1(O)C(=O)OCn2c1cc1c(c2=O)Cc2cc3ccccc3nc2-1 ; CC[C@@]1(OC(=O)OC(C)[C@]23O[C@](COC(=O)C(C)(C)C)(C=C2Oc2ccccc2)[C@@H]2C(=O)N(CCOC(=O)C(C)(C)Br)C(=O)[C@@H]23)C(=O)OCc2c1cc1n(c2=O)Cc2cc3ccccc3nc2-1]

TLC (5% methanol/dichloromethane): R_{f}=0.57

¹H NMR (400 MHz, CDCl₃) δ: 8.39 (s, 1H), 8.07 (d, J=8.5 Hz, 1H), 7.92 (dd, J=8.3, 1.3 Hz, 1H), 7.81 (ddd, J=8.4, 6.9, 1.2 Hz, 1H), 7.69-7.62 (m, 1H), 7.35 (s, 1H), 7.25-7.19 (m, 2H), 7.13-7.07 (m, 1H), 6.85 (dd, J=7.6, 1.6 Hz, 2H), 5.70 (d, J=17.2 Hz, 1H), 5.50 (q, J=6.5 Hz, 1H), 5.41 (d, J=17.3 Hz, 1H), 5.28 (s, 2H), 4.93 (s, 1H), 4.56 (Abq, ΔV_{AB}=120 Hz, J_{AB}=12.7 Hz, 2H), 3.90-3.79 (m, 2H), 3.73-3.63 (m, 2H), 3.26-3.13 (m, 1H), 3.04-2.92 (m, 1H), 2.35 (dq, J=14.9, 7.4 Hz, 1H), 2.19 (dq, J=14.8, 7.5 Hz), 1.71 (s, 3H), 1.69 (s, 3H), 1.54 (d, J=6.6 Hz, 3H), 1.21 (s, 9H), 1.01 (t, J=7.5 Hz, 3H).

¹³C{¹H} NMR (101 MHz, CDCl₃) δ: 177.8, 173.5, 172.6, 171.2, 167.3, 162.1, 157.3, 154.5, 153.0, 152.5, 148.8, 146.6, 145.4, 131.4, 131.0, 130.1, 129.4, 128.7, 128.4, 128.24, 128.22, 125.9, 120.8, 119.4, 101.3, 95.7, 90.2, 88.6, 78.2, 71.8, 67.4, 62.3, 62.2, 55.5, 51.4, 50.1, 47.9, 39.0, 36.9, 32.1, 30.53, 30.46, 27.2, 15.7, 7.8.

HRMS (ESI, m/z): calcd for [C₄₉H₄₉BrN₃O₁₄⁺] (M+H)⁺982.2392, found 982.2408.

Representative procedure for the synthesis of poly(2-(methylsulfinyl)ethyl acrylate) (PMSEA) polymers. PMSEA polymers were synthesized by controlled radical polymerization following an adaptation of the procedure by Nguyen et aL. (7). A flame-dried Schlenk flask was charged with freshly cut 20 G copper wire (2 cm), initiator 1-CoumNH₂ (3.5 mg, 0.0040 mmol), dimethyl sulfoxide (DMSO) (5 mL), and 2-methylsulfinyl ethyl acrylate (2.46 g, 15.2 mmol). The flask was sealed and the solution was degassed via three freeze-pump-thaw cycles, then backfilled with nitrogen and warmed to room temperature. Me₆TREN (5.0 μL, 0.019 mmol) was added via microsyringe and the reaction was stirred at room temperature. Upon completion of the polymerization after 6 h, the flask was opened to atmosphere and diluted with a minimal amount of methanol. The polymer was precipitated 3× into room temperature diethyl ether, dried under vacuum, dialyzed in water (10 mL in 4 L, 5×) and finally lyophilized to afford PMSEA-CoumNH₂ as a foamy white solid (822 mg, 33%). Mₙ=260 kg/mol, Ð=1.47.

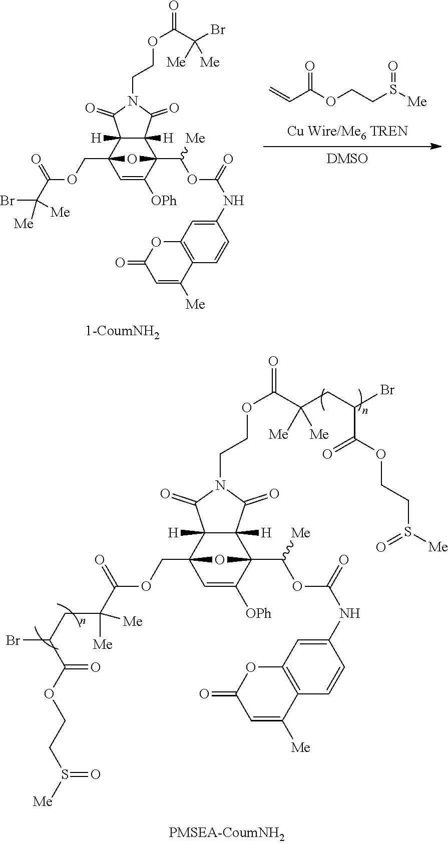

### Example 4: Procedure for Ultrasonication Experiments with an Immersion Probe

An oven-dried sonication vessel was fitted with rubber septa, placed onto the sonication probe, and allowed to cool under a stream of dry nitrogen. The vessel was charged with a solution of the polymer in either MilliQ water or 3:1 acetonitrile/water (2 mg/mL, 20 mL total volume) and submerged in an ice bath. The solution was sparged with nitrogen for 1 h prior to sonication, and a nitrogen headspace was maintained throughout the experiment. Pulsed ultrasound (1 s on/1 s off, 20% amplitude, 20 kHz, 9.04 W/cm²) was then applied to the system. “Sonication time” is defined as the duration of exposure to sonication, which is 50% of experimental or “clock” time under 1 s on/1 s off pulsation. The solution temperature during sonication was measured to be ˜10° C. by an internal temperature probe. Aliquots taken from the sonicated solution were filtered through a 0.45 μm PTFE syringe filter prior to analysis.

### Example 5: Procedure for Experiment with Focused Ultrasound (FUS)

FUS setup. A 2-mL microcentrifuge tube was placed on a 3D-printed sample stage, and the center position of the tube was determined using an L22-14vX 128-element Verasonics imaging probe. The tube was removed and exchanged with a needle hydrophone (Onda, HNR-0500) situated in the exact position as the previously registered center of the tube. A 330 kHz focused ultrasound transducer (Precision Acoustics, H115) was mounted on a computer-controlled 3D translatable stage (Velmex) orthogonally to the Verasonics probe. A previously reported MATLAB program was used to automatically scan and align the transducer's focus at the center of the microcentrifuge tube, according to the feedback from the needle hydrophone, which reports the acoustic pressure (5). To calibrate pressure, the needle hydrophone was placed inside and at the center of a plastic microcentrifuge tube to account for the attenuation of ultrasound wave through the plastic tube.

Spatial peak-temporal average acoustic intensity (Iₛₚₜₐ) is calculated with eq.

$$\\ \begin{matrix}{I}_{spta} = \frac{{p}^{{}_{} 2}}{2 ρ c} \cdot DC & ( S 1 )\end{matrix}$$

where p is the peak negative pressure (PNP) of ultrasound, ρ is the density of aqueous media, c is the speed of sound in aqueous media, DC is the duty cycle applied in ultrasound treatment.

Temperature calibration. Temperature change inside the microcentrifuge tube was monitored by a temperature probe, which is inserted inside the tube. The volume of sample inside the microcentrifuge tube was maintained at 800 μL in all experiments. Once 330 or 916 kHz FUS was turned on, the temperature inside the tube was recorded until it reached equilibrium. The measured temperature remained relatively constant when the temperature probe was moved within the tube while the ultrasound remained on. Different ultrasound conditions, including pressure, duty cycle, cycle number in each emitting pulse were systematically investigated to find the upper limit of ultrasound conditions for maintaining biocompatible temperatures. Temperature increment in different sample container (plastic microcentrifuge tubes vs tissue-mimicking 1% agarose phantom) was also investigated with the same ultrasound conditions. As demonstrated with the biocompatible FUS conditions, the temperature increase is higher for the sample in the microcentrifuge tube because the plastic walls absorb more ultrasound than agarose gel or water, resulting in increased heat dissipation at the interface.

### Example 6: Characterization of Aminocoumarin Release Using Photoluminescence

### Spectroscopv

Aliquots from either the sonication (immersion probe) or FUS experiments were filtered through 0.45 μm syringe filters and added to either a quartz microcuvette or a 96-well microplate for photoluminescence measurements. The samples were then analyzed by fluorescence spectroscopy on either a Shimadzu fluorimeter or a Tecan Spark microplate reader at room temperature. Emission spectra were recorded using an excitation wavelength of 365 nm. The concentration of released aminocoumarin (CoumNH₂) was determined from calibration curves constructed on both the Shimadzu fluorimeter and the Tecan Spark microplate reader.

Aminocoumarin Release with Immersion Probe Sonication. As expected, efficiency of CoumNH₂ release from PMSEA-CoumNH₂ (2 mg/mL in water) increases as a function of exposure time to sonication. Maximum release of CoumNH₂ was observed after 120 min sonication in water (44%). In contrast to the significant release triggered from PMSEA-CoumNH₂ containing a chain-centered mechanophore, sonication of chain-end functional polymer Control-CoumNH₂ (2 mg/mL in water) does not result in significant release of CoumNH₂, supporting that release occurs via a mechanochemical process.

Aminocoumarin Release with Biocompatible FUS. While typical biocompatible FUS experiments were performed for only 10 min, CoumNH₂ release efficiency was observed to increase with additional insonation of PMSEA-CoumNH₂ (2 mg/mL in water, 1.4 nM GVs). In contrast, exposure of chain-end functional control polymer Control-CoumNH₂ (2 mg/mL in water) to biocompatible FUS resulted in insignificant release of CoumNH₂. Likewise, no significant release of CoumNH₂ was observed from either PMSEA-CoumNH₂ or Control-CoumNH₂ over 17 h upon heating to 50° C., further supporting that release under FUS is mechanochemically triggered. Insignificant release of CoumNH₂ was also observed from PMSEA-CoumNH₂ when peak negative pressure (PNP) was <1.3 MPa, despite collapse of GVs occurring under these conditions. The extent of CoumNH₂ release was also dependent on the concentration of GVs, whereby a higher concentration of GVs resulted in greater percent release of CoumNH₂, which can be attributed to an increased number of cavitation events and consequently a greater extent of mechanochemical activation. Furthermore, insonation of PMSEA-CoumNH₂ in the presence of pre-collapsed GVs (1.4 nM), wherein gas content inside the nanostructures and surrounding fluid is substantially depleted, resulted in insignificant CoumNH₂ release, demonstrating that the gas content within intact GVs is crucial for efficient mechanochemical activation. Finally, the extent of CoumNH₂ release varied insignificantly with polymer concentration between 2 and 8 mg/mL.

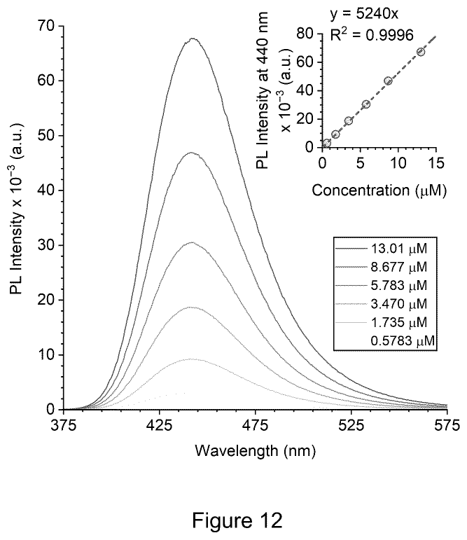

FIG. 12 illustrates construction of a calibration curve to determine the concentration of aminocoumarin (CoumNH₂) from fluorescence measurements on a Shimadzu fluorimeter in accordance with an embodiment. Emission spectra (λₑₓ=365 nm) and intensity at 440 nm for solutions of CoumNH₂ in water (with ≤0.1% DMSO for solubility) as a function of concentration. A linear regression of the data in the inset gives the calibration function y=5240x (R²=0.9996).

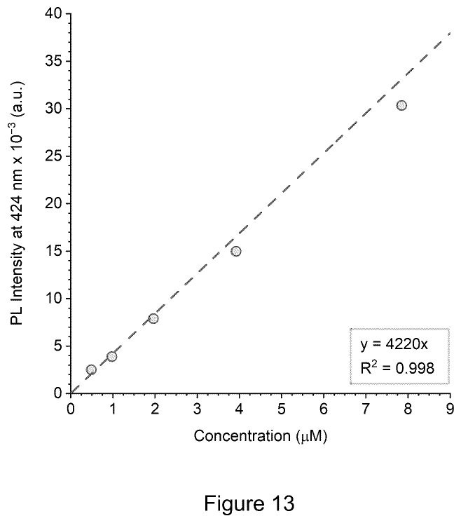

FIG. 13 illustrates a calibration curve to determine the concentration of aminocoumarin in accordance with an embodiment. Construction of a calibration curve for experimental determination of the concentration of aminocoumarin (CoumNH₂) using a Shimadzu fluorimeter. Emission intensity at 424 nm (λₑₓ=365 nm) for solutions of CoumNH₂ in 3:1 acetonitrile/methanol as a function of concentration. A linear regression of the data gives the calibration function y=4220x (R²=0.998).

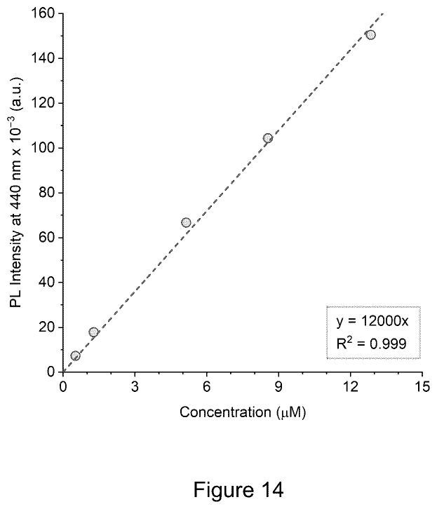

FIG. 14 illustrates a calibration curve to determine the concentration of aminocoumarin in accordance with an embodiment. Construction of a calibration curve for experimental determination of the concentration of aminocoumarin (CoumNH₂) using a Tecan Spark microplate fluorescence reader. Emission intensity at 440 nm (λₑₓ=365 nm) for solutions of CoumNH₂ in water as a function of concentration. A linear regression of the data gives the calibration function y=12000x (R²=0.999).

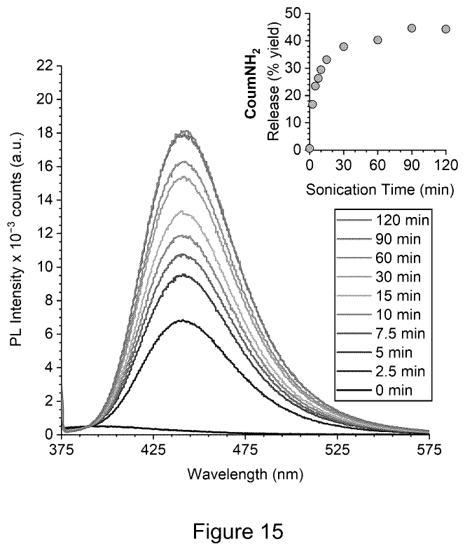

FIG. 15 illustrates the release of aminocoumarin as measured by photoluminescence in accordance with an embodiment. Time-dependent PL spectra (λₑₓ=365 nm) collected on a Shimadzu fluorimeter illustrate aminocoumarin release during sonication of PMSEA-CoumNH₂ using an immersion probe (2 mg/mL in water, 20 kHz, 9.04 W/cm²).

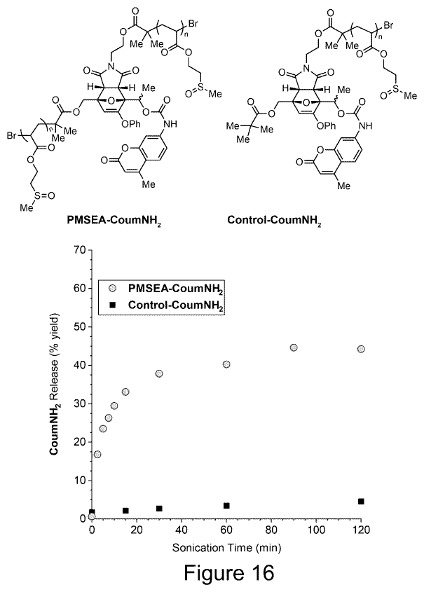

FIG. 16 illustrates the release of aminocoumarin as a function of sonication time in accordance with an embodiment. Release of aminocoumarin from PMSEA-CoumNH₂ and Control-CoumNH₂ as a function of sonication time quantified by PL measurements on a Shimadzu fluorimeter. Polymers were sonicated with an immersion probe (2 mg/mL in water, 20 kHz, 9.04 W/cm²).

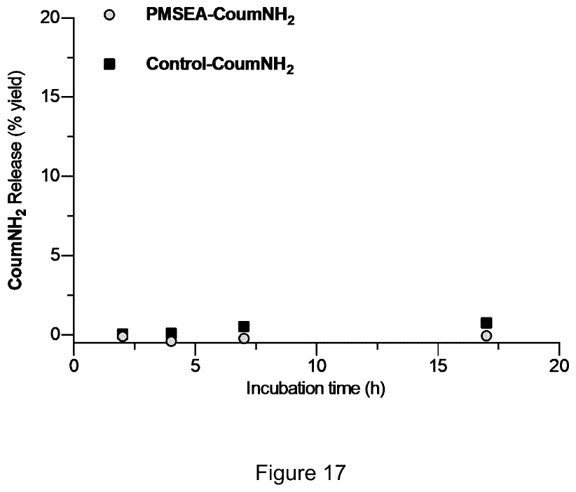

FIG. 17 illustrates the release of aminocoumarin in water at 50° C. as a function of incubation time in accordance with an embodiment. Insignificant release of CoumNH₂ from PMSEA-CoumNH₂ is observed at elevated temperatures. Polymer solutions (2 mg/mL in water) were incubated at 50° C. Time-dependent CoumNH₂ release was measured by PL measurements on a Tecan Spark microplate reader.

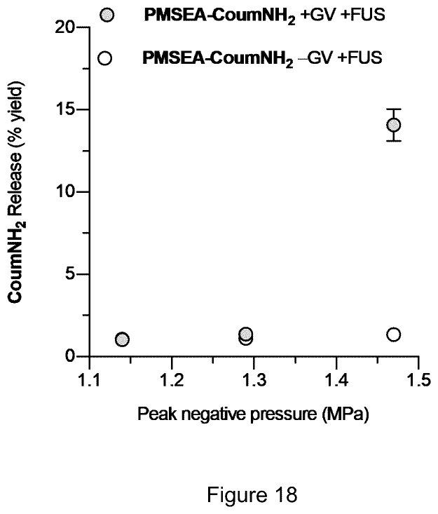

FIG. 18 illustrates the release of CoumNH₂ as a function of peak negative pressure with focused ultrasound in the presence and absence of gas vesicles in accordance with an embodiment. Pressure-dependent release of aminocoumarin (CoumNH₂) from PMSEA-CoumNH₂ (2 mg/mL in water) after FUS treatment. FUS was maintained at 3000 cycles each pulse, 4.5% duty cycle (equivalent to 5 Hz pulse repetition frequency), 10 min total insonation at various pressures. Release yield was measured by PL intensity at 440 nm (λₑₓ=365 nm) on a Tecan Spark microplate reader. For the samples containing GVs, the GV concentration is OD₅₀₀=12, corresponding to ˜1.4 nM. All measurements are based on an average of 3 replicates. Error bars represent standard deviation from three fluorescence measurements each on samples from three separate experiments (N=9).

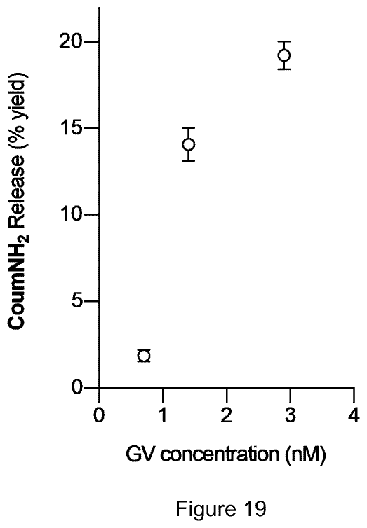

FIG. 19 illustrates the release of aminocoumarin as a function of GV concentration in accordance with an embodiment. Effect of GV concentration on release efficiency of CoumNH₂ from PMSEA-CoumNH₂ (2 mg/mL in water). FUS was maintained at 1.47 MPa peak negative pressure, 3000 cycles each pulse, 4.5% duty cycle (equivalent to 5 Hz pulse repetition frequency), 10 min total insonation. Release yield was measured by PL measurements on a Tecan Spark microplate reader. The GV concentrations were OD₅₀₀=6, 12, and 25 corresponding to ˜0.7, 1.4 and 2.9 nM. All measurements are based on an average of 3 replicates. Error bars represent standard deviation from one fluorescence measurement each on sample from three separate experiments (N=3).

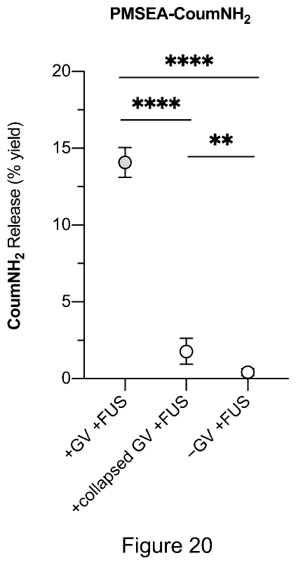

FIG. 20 illustrates the release of aminocoumarin under focused ultrasound conditions with and without intact GVs in accordance with an embodiment. Effect of GV gas content on release efficiency of CoumNH₂ from PMSEA-CoumNH₂ (2 mg/mL in water). FUS was maintained at 1.47 MPa peak negative pressure, 3000 cycles each pulse, 4.5% duty cycle (equivalent to 5 Hz pulse repetition frequency), 10 min total insonation. Release yield was measured by PL measurements on a Tecan Spark microplate reader. Experiments were performed with intact GVs, with pre-collapsed and degassed GVs, and without GVs. The GV concentration was OD₅₀₀=12, corresponding to ˜1.4 nM. All measurements are based on an average of 3 replicates. Error bars represent standard deviation from three fluorescence measurements each on samples from three separate experiments (N=9). Asterisks represent statistical significance by ordinary one-way ANOVA with multiple comparison (****P<0.0001; ***P<0.001; **P<0.01; NS, not significant).

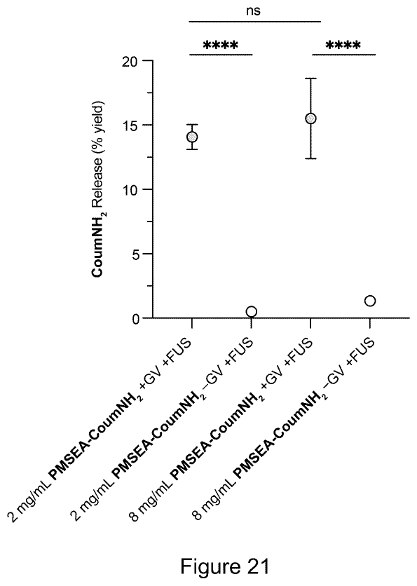

FIG. 21 illustrates the release of aminocoumarin at various polymer concentrations using focused ultrasound with and without GVs in accordance with an embodiment. Effect of polymer concentration on the release of CoumNH₂ from PMSEA-CoumNH₂ (2 or 8 mg/mL in water) after FUS treatment. FUS was maintained at 1.47 MPa peak negative pressure, 3000 cycles each pulse, 4.5% duty cycle (equivalent to 5 Hz pulse repetition frequency), 10 min total insonation. Release yield was measured by PL measurements on a Tecan Spark microplate reader. The GV concentration was OD₅₀₀=12, corresponding to ˜1.4 nM. All measurements are based on an average of 3 replicates. Error bars represent standard deviation from three fluorescence measurements each on samples from three separate experiments (N=9). Asterisks represent statistical significance by ordinary one-way ANOVA with multiple comparison (****P<0.0001; ***P<0.001; **P<0.01; NS, not significant).

### Example 7: Characterization of Camptothecin Release Using Liquid Chromatography

Several embodiments provide confirmation of CPT release via ¹H NMR spectroscopy and LCMS. Prior to mechanochemical activation studies, we first confirmed that camptothecin was released in polar protic solvent upon unmasking of the expected unstable furfuryl carbonate. To enable characterization, the furfuryl carbonate species was generated thermally from small molecule initiator 1-CPT. Masked furfuryl carbonate 1-CPT was dissolved in toluene-d₈ and analyzed by ¹H NMR spectroscopy before and after heating for 24 h at 100° C. Comparison to spectrum of the expected maleimide fragment confirmed significant thermal retro-Diels-Alder reaction occurred under these conditions. A drop of this solution was then diluted into 3:1 acetonitrile/methanol and analyzed by LCMS (gradient of 0-50% acetonitrile/water over 4 minutes). Comparison of the newly generated peak to a standard sample of pure camptothecin (CPT) reveals identical retention times, absorption spectra, and mass-to-charge ratios, supporting that CPT is released from the unmasked furfuryl carbonate upon exposure to polar protic solvent.

Calculation of HPLC Relative Response Factors (RRF). Three standard solutions with known concentrations of 4-methyl-2-oxo-2H-chromen-7-yl acetate internal standard (IS) and CPT were prepared and analyzed by HPLC equipped with a UV detector. The RRF is calculated from the HPLC results (C8 column, isocratic 30% acetonitrile/water) of the standard solution using eq. S2:

$$\\ \begin{matrix}RRF = \frac{Analyte Response Factor}{IS Response Factor} = \frac{( \frac{Analyte peak area}{Analyte concentration} )}{( \frac{IS peak area}{IS concentration} )} & ( S 2 )\end{matrix}$$

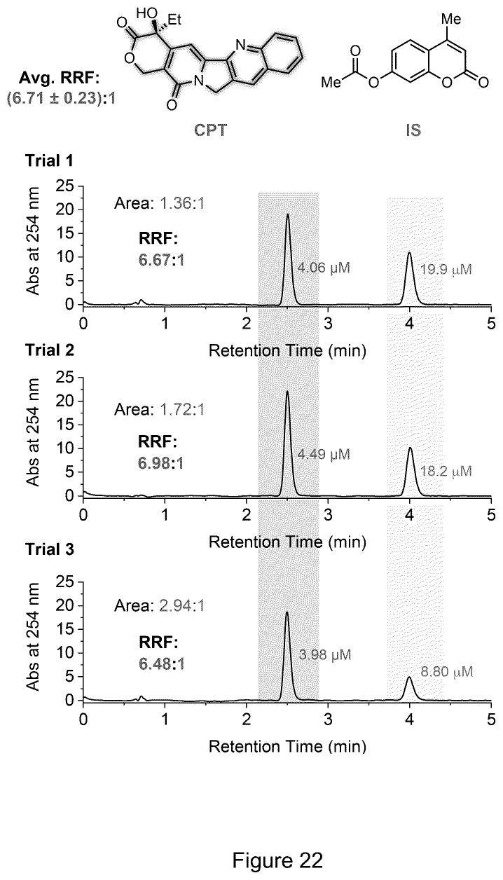

RRF at 254 nm was determined to be 6.71±0.23 for CPT:IS. FIG. 22 illustrates the relative response factor of analyte in accordance with an embodiment. The relative response factor (RRF) of analyte (CPT) versus internal standard (IS) was determined to be 6.71±0.23 from the average of three separate volumetric solutions.

Determination of the concentration of released CPT from polymers after FUS exposure. A solution of IS of known concentration was added to analyte solutions in a volumetric ratio of 1:3 (IS:analyte solution). The solution was then kept at room temperature and analyzed by HPLC (50 μL injection, C8 column, isocratic 30% acetonitrile/water) at various time intervals. The total concentration of released CPT was calculated using the relationship in eq. S3:

$$\\ \begin{matrix}Released CPT ( µM ) = \frac{Peak area of CPT}{Peak area of IS} * \frac{1}{RRF} * Concentration of IS ( µM ) * \frac{4}{3} & ( S 3 )\end{matrix}$$

No release of CPT is observed from aqueous solutions of PMSEA-CPT stored under ambient conditions for 2 months. Applying FUS (330 kHz, 3.6 W/cm², 1.47 MPa PNP, 4.5% duty cycle, 10 min) to PMSEA-CPT (4 mg/mL in water) triggers release of CPT (7.732±0.006%, average of three trials) in the presence of GVs (1.4 nM). No release is observed with FUS in the absence of GVs. Release of CPT is also not observed upon insonation of chain-end functional polymer Control-CPT in the presence of GVs (1.4 nM), supporting that mechanical force is responsible for activation. Finally, free CPT was confirmed to be stable upon exposure to FUS (10 min) in either the presence or absence of GVs (1.4 nM).

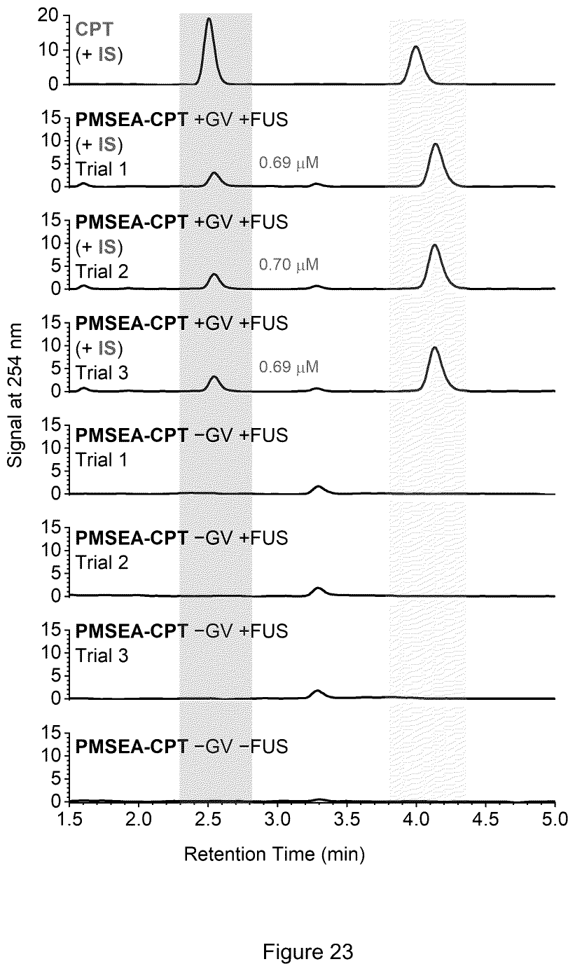

FIG. 23 illustrates HPLC chromatograms confirming selective cargo release in the presence of GVs using biocompatible focused ultrasound in accordance with an embodiment. Exposure of PMSEA-CPT (4 mg/mL in water) to biocompatible FUS (330 kHz, 3.6 W/cm², 1.47 MPa PNP, 4.5% duty cycle, 10 min) in the presence of GVs (1.4 nM) triggers the release of CPT as measured by HPLC (C8 column, isocratic 30% acetonitrile/water). Experiments were run in triplicate, and samples were diluted 3:1 with a stock solution of IS (72.8 μM). Concentrations were determined from integration of the peak corresponding to CPT relative to that of the internal standard and scaled by 4/3 to account for dilution. No IS was used in the experiments without GVs.

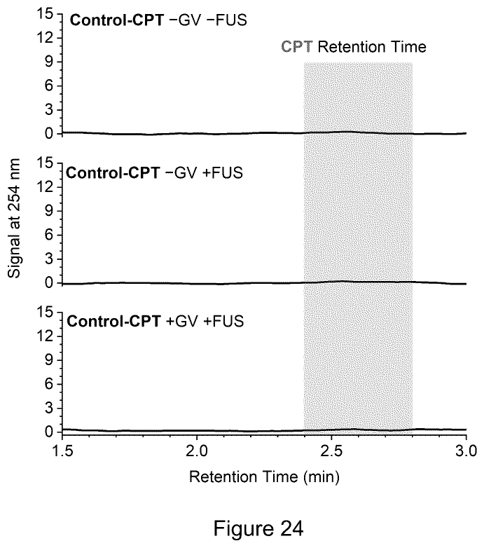

FIG. 24 illustrates HPLC chromatograms for control experiments in accordance with an embodiment. HPLC measurements demonstrating that exposure of chain-end functional polymer Control-CPT (4 mg/mL in water) to biocompatible FUS (330 kHz, 3.6 W/cm², 1.47 MPa PNP, 4.5% duty cycle, 10 min) in the presence or absence of GVs (1.4 nM) does not result in the release of CPT.

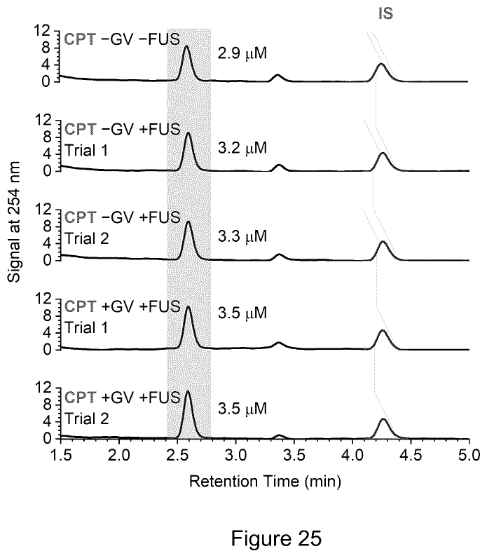

FIG. 25 illustrates HPLC chromatograms demonstrating stability of camptothecin under focused ultrasound conditions in accordance with an embodiment. HPLC measurements demonstrating stability of CPT upon exposure to biocompatible FUS (330 kHz, 3.6 W/cm², 1.47 MPa PNP, 4.5% duty cycle, 10 min) in the presence or absence of GVs (1.4 nM). Experiments were performed in duplicate on a sample of CPT in water (˜3 μM). After insonation, samples were diluted 3:1 with a stock solution of IS (40 μM) and concentrations were determined from integration of the peak corresponding to CPT relative to that of the internal standard and scaled by 4/3 to account for dilution.

### Example 8: Cytotoxicity Experiments

Several embodiments use MTT-based cell viability assay. Raji cells were used to evaluate the effectiveness of CPT release for potential chemotherapy. Raji cells were incubated in RPMI 1640 media, with 10% fetal bovine serum and 1% antibacterial Penicillin/Streptomycin (PenStrep) at 37° C. and 5% CO₂. The instructions provided by the manufacturer (Abcam) were followed to perform the MTT assay. Approximately 10,000 Raji cells suspended in 150 μL of media were seeded into each well of a 96-well plate. Subsequently, 50 μL of polymer sample (or pure CPT) at varying concentrations was added to corresponding wells. For calibration purposes, wells without cells and wells with only cells and media were also prepared, representing 0% and 100% cell viability, respectively. After 48 hours of incubation, cells were spun down inside the plate and media were carefully aspirated. Then 50 μL of serum free media and 50 μL of MTT solution were added to each well. Following a 3 h incubation, 150 μL MTT solution was added to each well. The plate was thoroughly mixed and shaken on an orbital shaker at 37° C. for 15 min. The absorbance at 590 nm was measured by a microplate reader (TECAN Spark) to quantify the cell viability in each well. Cell viability is calculated using eq. S4:

$$\\ \begin{matrix}Cell viability = \frac{A - {A}_{media only}}{{A}_{cell only} - {A}_{media only}} & ( S 4 )\end{matrix}$$

where A, A_{media only}, and A_{cell only} represent absorbance of the sample, control with media only, and control with only cell and media but no analyte.

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

As used herein, the singular terms “a,” “an,” and “the,” may include plural referents unless the context clearly dictates otherwise. Reference to an object in the singular is not intended to mean “one and only one” unless explicitly so stated, but rather “one or more.”

As used herein, the terms “approximately,” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to +0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

What is claimed is:

1. A method for activating a mechanochemical reaction comprising:
applying ultrasound to a solution at least containing a concentration of at least one polymer and a plurality of gas-filled structures;
wherein each of the at least one polymer comprises a polymer chain functionalized with at least one mechanophore; and
wherein the ultrasound is applied such that the plurality of gas-filled structures collapses thereby transducing the ultrasound to a mechanical force, such that the mechanical force activates the mechanochemical reaction to produce a functional response from the at least one mechanophore.

2. The method of claim 1, wherein the at least one mechanophore is selected from the group consisting of: a mechanochromic mechanophore, a mechanophore with a cargo molecule, a mechanophore that changes electrical conductivity, and a mechanophore that reveals at least one reactive group.

3. The method of claim 2, wherein the function response from the at least one mechanophore is selected from the group consisting of: changing color, changing luminescence, releasing the cargo molecule, changing electrical conductivity, and revealing the at least one reactive group.

4. The method of claim 2, wherein the at least one mechanophore is selected from the group consisting of: spiropyran, a derivative of spiropyran, naphthopyran, benzopyran, rhodamine, oxazine, triarylmethane, diarylbibenzofuranone, a derivative of cyclobutane, benzocyclobutene, dihalocycloproprane, a Diels-Alder adduct, a beta-lactam, ladderane, and masked 2-furylcarbinol.

5. The method of claim 1, wherein the at least one mechanophore is attached to the polymer chain via: a covalent bond, bioconjugation, or click chemistry.

6. The method of claim 1, wherein the at least one mechanophore comprises at least one cargo molecule selected from the group consisting of: a macromolecule, a small molecule, a micromolecule, an organic molecule, an inorganic molecule, an amino acid, a polypeptide, a protein, a nucleic acid, a DNA, an RNA, a monosaccharide, a polysaccharide, and any combinations thereof.

7. The method of claim 1, wherein the at least one mechanophore comprises at least one cargo molecule selected from the group consisting of: a drug, a chemotherapeutic drug, a catalyst, a fluorescent molecule, a fluorescent probe, a fluorophore, and a luminescent molecule.

8. The method of claim 1, wherein the ultrasound is a focused ultrasound.

9. The method of claim 1, wherein the polymer chain is poly(2-(methylsulfinyl)ethyl acrylate), the mechanophore is a masked 2-furylcarbinol mechanophore, and an anticancer drug camptothecin is covalently attached to the mechanophore.

10. The method of claim 1, wherein the solution is selected from the group consisting of: an aqueous solution, an organic solution, a buffer solution, an intercellular environment, an intracellular environment, an in situ environment, an in vitro environment, an in vivo environment, a physiological environment, and a clinically relevant environment.

11. The method of claim 1, wherein each of the plurality of gas-filled structures is selected from the group consisting of: a gas vesicle, a natural gas vesicle, a synthetic gas vesicle, a microbubble, and any combinations thereof.

12. The method of claim 1, wherein each of the plurality of gas-filled structures comprises a gas selected from the group consisting of: a non-reactive gas, an inert gas, air, nitrogen, carbon dioxide, helium, argon, neon, xenon, and any combinations thereof.

13. The method of claim 1, wherein each of the plurality of gas-filled structures has an average diameter from 50 nm to 10 microns.

14. The method of claim 1, wherein each of the plurality of gas-filled structures has an average length from 50 nm to 10 microns.

15. The method of claim 1, wherein the plurality of gas-filled structures comprises a plurality of gas vesicles, and the plurality of gas vesicles has an average diameter from 45 nm to 250 nm and an average length from 100 nm to 600 nm.

16. The method of claim 1, wherein the ultrasound causes a temperature increase of the solution less than or equal to 5° C.

17. The method of claim 1, wherein the ultrasound has an acoustic intensity Iₛₚₚₐ (spatial-peak-pulse-average) less than or equal to 1000 W/cm².

18. The method of claim 1, wherein the ultrasound has an acoustic intensity Iₛₚₜₐ (spatial-peak-time-average) less than or equal to 50 W/cm².

19. The method of claim 1, wherein the ultrasound has an acoustic intensity Iₛₚₚₐ less than or equal to 80 W/cm² and Iₛₚₜₐ less than or equal to 4 W/cm².

20. A method for delivering one or more cargo molecules comprising:
applying focused ultrasound to an aqueous environment at least containing a concentration of at least one polymer and a plurality of gas-filled structures;
wherein each of the at least one polymer comprises a polymer chain functionalized with at least one mechanophore, and wherein the at least one mechanophore comprises at least one cargo molecule; and
wherein the focused ultrasound is applied such that the plurality of gas-filled structures collapses thereby transducing the focused ultrasound to a mechanical force, such that the mechanical force activates the at least one mechanophore to release the at least one cargo molecule into the aqueous environment.

21. The method of claim 20, wherein the one or more cargo molecules is delivered with temporal and spatial control.

22. A method for drug delivery comprising:
applying focused ultrasound to an aqueous environment at least containing a concentration of at least one polymer and a plurality of gas-filled structures;
wherein each of the at least one polymer comprises a polymer chain functionalized with at least one mechanophore, and wherein the at least one mechanophore comprises at least one drug molecule; and
wherein the focused ultrasound is applied such that the plurality of gas-filled structures collapses thereby transducing the focused ultrasound to a mechanical force, such that the mechanical force activates the at least one mechanophore to release the at least one drug molecule into the aqueous environment.

23. The method of claim 22, wherein the at least one drug molecule is selected from the group consisting of: an anticancer drug, a chemotherapeutic drug, a small molecule drug, a biologic drug, a macromolecule drug, and a micromolecule drug.
